(19) United States
(12) Patent Application Publication
(10) Pub. No.: US 2022/0151496 A1
(43) Pub. Date: May 19, 2022

(54) DEVICE AND METHOD FOR ANALYZING OPTOACOUSTIC DATA, OPTOACOUSTIC SYSTEM AND COMPUTER PROGRAM

(71) Applicants: iThera Medical GmbH, München (DE); Friedrich-Alexander-Universität Erlangen-Nürnberg, Erlangen (DE)

(72) Inventors: Maximilian Waldner, Nürnberg (DE); Ferdinand Knieling, Forchheim (DE); Adrian Regensburger, Erlangen (DE); Jing Claussen, München (DE)

(21) Appl. No.: 17/439,383

(22) PCT Filed: Mar. 12, 2020
(86) PCT No.: PCT/EP2020/056694

§ 371 Date: Sep. 14, 2021

(30) Foreign Application Priority Data
Mar. 15, 2019 (EP) 19163304.9

(51) Int. Cl. A61B 5/00; A61K 31/573

(52) U.S. Cl. CPC A61B 5/0095; A61B 5/4519; A61B 2576/02; A61K 31/573; A61B 5/742

(57) ABSTRACT

The invention relates to a device and a method for analyzing optoacoustic data, an optoacoustic system for generating and analyzing optoacoustic data and a computer program. The device for analyzing optoacoustic data according to a first aspect of the invention comprises a data processing unit configured to determine a spatial distribution of at least one first value, which relates to concentration of collagen in a tissue comprising at least one of a muscle tissue, connective tissue, organ, tendon and/or pathogenic (fibrotic) tissue, based on optoacoustic data relating to acoustic waves generated in the tissue in response to irradiating the tissue with time-varying electromagnetic radiation at two or more different irradiation wavelengths, derive at least one second value from the spatial distribution of the at least one first value, the at least one second value corresponding to or being derived from at least one distribution parameter characterizing the spatial distribution of the at least one first value within a region of interest of the spatial distribution of the at least one first value, and provide the at least one second value and/or diagnostic information derived from the at least one second value for further use, in particular for displaying the at least one second value and/or diagnostic information on a display unit.

The present invention relates to a device and a method for analyzing optoacoustic data, an optoacoustic system for generating and analyzing optoacoustic data and a computer program.

Optoacoustic imaging is based on the photoacoustic effect, according to which ultrasonic waves are generated due to absorption of electromagnetic radiation by an object, e.g. a biological tissue, and a subsequent thermoelastic expansion of the object. Excitation radiation, for example laser light or radiofrequency radiation, can be pulsed radiation with short pulse durations or continuous radiation with varying amplitude or frequency.

It is an object of the invention to provide a device, a method, an optoacoustic system and a computer program allowing for an improved analysis of optoacoustic data, in particular regarding diagnostic purposes.

This object is achieved by a device, a method, an optoacoustic system and a computer program according to the independent claims.

A device for analyzing optoacoustic data according to a first aspect of the invention comprises a data processing unit configured to determine a spatial distribution of at least one first value (e.g. optoacoustic collagen signal in arbitrary units [a.u.]), which relates to a concentration of collagen in a tissue comprising at least one of a muscle tissue, connective tissue, organ, tendon and/or pathogenic (e.g. fibrotic) tissue, based on optoacoustic data (e.g. detector signals) relating to acoustic waves generated in the tissue in response to irradiating the tissue with time-varying electromagnetic radiation at two or more different irradiation wavelengths, derive at least one second value (e.g. collagen_mean, collagen_max, collagen_mean/collagen_max) from the spatial distribution of the at least one first value, the at least one second value corresponding to or being derived from at least one distribution parameter (e.g. mean/max) characterizing the spatial distribution of the at least one first value within a region of interest (ROI) of the spatial distribution of the at least one first value, and provide the at least one second value and/or diagnostic information derived from the at least one second value for further use, in particular for displaying the at least one second value and/or diagnostic information on a display unit.

An optoacoustic system for generating and analyzing optoacoustic data according to a second aspect of the invention comprises an irradiation unit configured to irradiate a tissue comprising muscle tissue with electromagnetic radiation at two or more different irradiation wavelengths, said electromagnetic radiation having a time-varying, in particular pulsed, intensity, a detection unit configured to detect acoustic waves generated in the tissue in response to irradiating the tissue with the electromagnetic radiation at the different irradiation wavelengths and to generate according optoacoustic data, and a device for analyzing optoacoustic data according to the first aspect of the invention.

A method for analyzing optoacoustic data according to a third aspect of the invention comprises the following steps: determining a spatial distribution of at least one first value (e.g. an optoacoustic collagen signal in arbitrary units [a.u.]), which relates to a concentration of collagen in tissue comprising at least one of a muscle tissue, connective tissue, organ, tendon and/or pathogenic (e.g. fibrotic) tissue, based on optoacoustic data (e.g. detector signals) relating to acoustic waves generated in the tissue in response to irradiating the tissue with time-varying electromagnetic radiation at two or more different irradiation wavelengths, deriving at least one second value (e.g. collagen_mean, collagen_max, collagen_mean/collagen_max) from the spatial distribution of the at least one first value, the at least one second value corresponding to or being derived from at least one distribution parameter (e.g. mean/max) characterizing the spatial distribution of the at least one first value within a region of interest of the spatial distribution of the at least one first value, and providing the at least one second value and/or diagnostic information derived from the at least one second value for further use, in particular displaying the at least one second value and/or diagnostic information on a display unit.

A method of treatment of a muscle disorder, in particular Duchenne muscular dystrophy (DMD), comprises the following steps:
determining a spatial distribution of at least one first value (e.g. optoacoustic collagen signal in arbitrary units [a.u.]), which relates to a concentration of collagen in a tissue comprising at least one of a muscle tissue, connective tissue, organ, tendon and/or pathogenic (e.g. fibrotic) tissue, of a subject, based on optoacoustic data (e.g. detector signals) relating to acoustic waves generated in the tissue in response to irradiating the tissue with time-varying electromagnetic radiation at two or more different irradiation wavelengths,
deriving at least one second value (e.g. collagen_mean, collagen_max, collagen_mean/collagen_max) from the spatial distribution of the at least one first value, the at least one second value corresponding to or being derived from at least one distribution parameter (e.g. mean/max) characterizing the spatial distribution of the at least one first value within a region of interest of the spatial distribution of the at least one first value,
outputting, in particular displaying on a display unit, the at least one second value and/or diagnostic information, which has been derived from the at least one second value by comparing the at least one second value with at least one predefined reference value and which relates to the presence or absence and/or likelihood of presence or absence of a muscle disorder, in particular Duchenne muscular dystrophy (DMD), in the tissue of the subject, and
treating the subject, in particular with a medication, preferably by administering a medication to the subject, depending on the output at least one second value and/or diagnostic information.

Aspects of present disclosure are preferably based on the approach of providing optoacoustic data, in particular single-wavelength optoacoustic images, which are or were obtained by irradiating a tissue, in particular a muscle tissue, with time-varying electromagnetic radiation at two or more different irradiation wavelengths and detecting acoustic waves generated in the tissue in response to irradiating the tissue. A spatial distribution of a first value, in particular an image depicting the first value, relating to a concentration of collagen in the tissue is determined based on the optoacoustic data, in particular on the single-wavelength optoacoustic images, obtained at the two or more different irradiation wavelengths. Further, at least one second value is derived for a region of interest (ROI) in the spatial distribution of the first value by determining at least one distribution parameter characterizing the spatial distribution of the first value within the ROI. Preferably, the at least on distribution parameter comprises a mean value corresponding to an average, e.g. arithmetic or geometric mean, a median (value separating the higher half from the lower half of the first values within the ROI) or mode (value that appears most often within the ROI) of the first values within the ROI and/or a maximum value corresponding to the highest first value within the ROI. Alternatively or additionally, the distribution parameter may comprise other statistical parameters, e.g. a dispersion parameter such as variance, standard deviation and/or interquartile range. Alternatively or additionally, the at least one second value can be derived from the at least on distribution parameter, e.g. from a mean and maximum value. The at least one second value and/or a diagnostic information which is derivable from the at least one second value is provided for further use, in particular by displaying the at least one second value and/or diagnostic information on a display unit.

Surprisingly it was found that the at least one second value obtained in this way can be used to infer the presence or absence of a muscle disorder, in particular Duchenne muscular dystrophy (DMD), in the tissue.

In summary, the invention allows for an improved analysis of optoacoustic data, in particular regarding diagnostic purposes such as diagnosing, monitoring and/or treating DMD.

Preferably, the diagnostic information can be derived from the at least one second value by comparing the at least one second value with at least one predefined reference value, which has been derived from a control cohort, e.g. healthy subjects, subjects (patients) without treatment and/or subjects (patients) in remission.

Alternatively or additionally, the diagnostic information can be derived from the at least one second value by comparing the at least one second value with at least one predefined reference value, which has been derived for the subject at an earlier point of time, e.g. prior to administering the medication to the subject. In this way, possible changes due to the treatment with respect to a point of time prior to beginning the treatment and/or a point of time during the treatment can be monitored.

Preferably, the at least one predefined reference value corresponds to at least one second value, which has been derived from a control cohort or for the subject, respectively, using the device, method and/or system according to the aspects of the invention.

Alternatively or additionally, the at least one predefined reference value can be derived by considering clinical tests, like blood creatine kinase values, or physiological tests, like the 6-minute walk test (6-MWT) or muscular strength tests of the control cohort or the subject, respectively.

Alternatively or additionally, the at least one predefined reference value can be derived by considering other modalities, like MRI or sonography. Preferably, treating the subject comprises administering corticosteroids and/or Spinraza® (Nusinersen) and/or Zolgensma® (Onasemnogene abeparvovec) to the subject and/or undergoing a gene therapy. Drug treatment with corticosteroids can help increase muscle strength and slow progression. Spinraza® (Nusinersen) and Zolgensma® (Onasemnogene abeparvovec) are prescription medicines used to treat spinal muscular atrophy (SMA) in pediatric and adult patients and are preferably administered by injection therapy. Further gene therapy may comprise treatment with, e.g., of the invention Exondys 51® (eteplirsen or AVI-4658).

Alternatively or additionally, treating the subject may comprise and/or be based on at least one of the following:
Utrophin production: Utrophin is a protein similar to dystrophin that is not affected by muscular dystrophy. If utrophin production is upregulated, the disease may be halted or slowed.
Altering protein production: If the dystrophin gene is being read by protein synthesis machinery and it reaches a mutation, it stops and does not complete the protein. Drugs are preferred that cause the protein-making equipment to skip the mutated content and still continue to create dystrophin.
Drugs to delay muscle wasting: Rather than targeting the genes behind muscular dystrophy, it is possible to slow the inevitable muscle wasting. Muscles, in standard circumstances, can repair themselves. Research into controlling or increasing these repairs showed benefits for people with muscular dystrophy.
Stem cell research: Muscle stem cells capable of producing the lacking dystrophin protein are used, in particular inserted. Current projects are looking at the most useful type of cells to use and ways in which they could be delivered to skeletal muscle.
Myoblast transplantation: During the early stages of muscular dystrophy, myoblasts (also called satellite cells) repair and replace faulty muscle fibers. As the myoblasts become exhausted, the muscles are slowly turned into connective tissue. Preferably, modified myoblast cells are inserted into muscles to take over from the exhausted natural myoblasts.

A method of monitoring treatment of a muscle disorder, in particular Duchenne muscular dystrophy (DMD), comprises the following steps:
treating a subject with a medication, preferably by administering a medication to the subject, and
deriving, preferably after a pre-defined period of time after treating the subject with the medication, at least one second value and/or deriving diagnostic information from the at least one second value, and outputting, in particular displaying on a display unit, the at least one second value and/or diagnostic information by using the device and/or system and/or method according to the first and/or second and/or third aspect of the invention.

A computer program product according to yet another aspect of the invention is configured to cause a computer to execute at least one of the methods described above.

Preferably, treatment of the subject can help prevent or reduce problems in the joints and spine to allow the subject with muscular dystrophy to remain mobile as long as possible. In general, treatment options include but are not limited to medications, physical and occupational therapy, and surgical and other procedures.

Preferably, treating the subject with a medication depending on the output at least one second value and/or diagnostic information includes, but is not limited to
selecting the medication and/or an active agent of the medication depending on the at least one second value and/or diagnostic information,
selecting the dose of an active agent contained in the medication depending on the at least one second value and/or diagnostic information,
selecting time and/or duration of administering the medication depending on the at least one second value and/or diagnostic information.

Preferably, treating the subject with a medication comprises administering at least corticosteroids or gene therapies, such as Deflazacort and/or Ataluren (Translarna™, PTC Therapeutics Inc.) and/or Spinraza® (Nusinersen) and/or Zolgensma® (Onasemnogene abeparvovec) or other approved drugs.

Alternatively or additionally, treating the subject may comprise at least one of:
administering Eteplirsen (Exondys 51®),
administering Corticosteroids, such as prednisone, and/or
heart medications, such as angiotensin-converting enzyme (ACE) inhibitors or beta blockers.

Preferably, within the context of present disclosure, the terms “optoacoustic” and “photoacoustic” are used synonymously.

Preferably, the spatial distribution of the at least one first value is a two-dimensional or three-dimensional spatial distribution of the at least one first value.

Preferably, the at least one second value corresponding to or being derived from at least one of the following distribution parameters: a mean value of the spatial distribution of the at least one first value within the region of interest, and/or a maximum value of the spatial distribution of the at least one first value within the region of interest.

Preferably, the at least one second value being derived from the mean value and the maximum value of the spatial distribution of the at least one first value within the region of interest.

Preferably, the at least one second value corresponds to a ratio between the mean value and the maximum value of the spatial distribution of the at least one first value within the region of interest.

Preferably, the data processing unit being further configured to derive the diagnostic information from the at least one second value by comparing the at least one second value with at least one predefined reference value.

Preferably, the derived diagnostic information relates to the presence or absence and/or likelihood of presence or absence of a muscle disorder, in particular Duchenne muscular dystrophy (DMD), in the tissue.

Preferably, the data processing unit being further configured to reconstruct an ultrasound image of the tissue based on ultrasound data (detector signals) relating to ultrasound waves reflected by the tissue in response to ultrasound waves impinging on the tissue, and provide the ultrasound image of the tissue for displaying the ultrasound image of the tissue on a display unit.

Preferably, the device further comprises a display unit configured to display information, wherein the data processing unit is further configured to control the display unit to display the spatial distribution of the at least one first value, which relates to a concentration of collagen in the tissue, and/or the ultrasound image of the tissue and/or the at least one second value and/or the diagnostic information derived from the at least one second value.

Preferably, the data processing unit being further configured to merge the spatial distribution of the at least one first value, which relates to a concentration of collagen in the tissue, and the ultrasound image of the tissue to obtain a merged optoacoustic-ultrasound image of the tissue, and to control the display unit to display the merged optoacoustic-ultrasound image of the tissue.

Preferably, the display unit and/or the data processing unit being further configured to enable a user to select the region of interest (ROI), within which the at least one second value is derived from the spatial distribution of the at least one first value, in the displayed spatial distribution of the at least one first value and/or in the displayed merged optoacoustic-ultrasound image of the tissue.

Preferably, the data processing unit is configured to receive the optoacoustic data from a detection unit of an optoacoustic imaging system and/or the data processing unit comprises an interface being configured to receive the optoacoustic data from a detection unit of an optoacoustic imaging system.

Preferably, the irradiation unit is configured to irradiate the tissue with electromagnetic radiation at two or more different irradiation wavelengths being in a wavelength range between 660 nm and 1300 nm, preferably between 680 nm and 1100 nm.

Alternatively or additionally, the objects and/or advantages disclosed herein may be preferably achieved by an optoacoustic system, an optoacoustic device, a method and a computer program according to at least one of the following aspects a) to z).

a. An optoacoustic system comprising

a probe, in particular a handheld probe, comprising
an irradiation unit configured to irradiate a tissue, in particular comprising muscle tissue, with electromagnetic radiation at two or more different irradiation wavelengths (λₖ, k=1 . . . K), said electromagnetic radiation having a time-varying, in particular pulsed, intensity, and
a detection unit comprising a concave, in particular spherically shaped, matrix array of transducer elements configured to detect acoustic waves generated in the tissue in response to irradiating the tissue with the electromagnetic radiation at the different irradiation wavelengths while the probe is being moved, in particular translated and/or rotated, to a plurality of different probe positions, in particular including different locations and/or orientations of the probe, relative to the tissue, and

a processing unit configured
to reconstruct, in particular 3D, optoacoustic images (also referred to as “single-wavelength optoacoustic images” or “single-wavelength MSOT images”) based on the acoustic waves which were detected at the different irradiation wavelengths (λₖ, k=1 . . . K) and probe positions so as to obtain, for each of the different irradiation wavelengths (λₖ, k=1 . . . K), a sequence of optoacoustic images (iₙ, n=1 . . . N), which were obtained for different probe positions,
to generate, for each of the different irradiation wavelengths, a compounded, in particular 3D, optoacoustic image (iₛ) from the sequence of optoacoustic images (iₙ, n=1 . . . N) by
i) determining a location (Tₙ, n=2 . . . N) and/or rotation (θₙ, n=2 . . . N) of at least one optoacoustic image (iₙ) of the sequence relative to an initial optoacoustic image (i₁) of the sequence, and
ii) combining the at least one optoacoustic image (iₙ) of the sequence with the initial optoacoustic image (i₁) of the sequence so as to obtain the compounded optoacoustic image (iₛ) by considering the determined location (Tₙ) and/or rotation (θₙ) of the at least one optoacoustic image (iₙ) of the sequence relative to the initial optoacoustic image (i₁) of the sequence, and
to determine at least one, in particular 3D, concentration image (also referred to as “MSP (multi-spectrally processed) image”) relating to a, in particular three-dimensional, spatial distribution of a concentration (also referred to as “first value”) of a substance, in particular a component and/or biomarker such as collagen, in the tissue based on the compounded optoacoustic images generated for the different irradiation wavelengths.

b. The optoacoustic system according aspect a), wherein the processing unit is further configured to determine the location (Tₙ) and/or rotation (θₙ) of the at least one optoacoustic image (iₙ) of the sequence relative to the initial optoacoustic image (i₁) of the sequence by

i) estimating translations (tₙ) and/or rotations (θₙ) between at least two consecutive optoacoustic images of the sequence and

ii) adding up the estimated translations (tₙ) and/or the estimated rotations (θₙ) between at least two consecutive optoacoustic images of the sequence.

c. The optoacoustic system according to aspect b), wherein the processing unit is further configured to estimate rotation angles (θₓ, θ_{y}, θ_{z}) of the rotation (θₙ) between two consecutive optoacoustic images (e.g. i₁, i₂) of the sequence by calculating a rotation correlation function (RCFₓ(θₓ), RCF_{y}(θ_{y}), RCF_{z}(θ_{z})) corresponding to a normalized cross-correlation function between 2D-Fourier transforms (I_{1, MIP, I}, I_{2, MIP, i}) of maximum intensity projections (MIPs) of the two consecutive optoacoustic images (i₁, i₂) along the x, y and z direction before and after rotation.

d. The optoacoustic systems according to aspect c), wherein the estimated rotation angles (θ′ₓ, θ′_{y}, θ′_{z}) correspond to the arguments of the maxima (arg max_θₓ{RCFₓ(θₓ)}, arg max_θ_{y}{RCF_{y}(θ_{y})}, arg max_θ_{z}{RCF_{z}(θ_{z})}) of the rotation correlation function (RCFₓ(θₓ), RCF_{y}(θ_{y}), RCF_{z}(θ_{z})).

e. The optoacoustic system any of the aspects b) to d), wherein the processing unit is further configured to estimate translation components (tₓ, t_{y}, t_{z}) of the translation (tₙ) between two consecutive optoacoustic images (e.g. i₁, i₂) of the sequence by calculating a phase-only correlation (PoC) corresponding to the inverse Fourier transform of a normalized phase correlation function (PCF) between the 3D-Fourier transform (I₁) of the first optoacoustic image (i₁) and the complex conjugate (I₂*) of the 3D-Fourier transform (I₂) of the second optoacoustic image (i₂).

f. The optoacoustic system according to aspect e), wherein the estimated translation components (t′ₓ, t′_{y}, t′_{z}) correspond to the arguments of the maxima (arg max__{x,y,z}{PoC(x,y,z)}) of the phase only correlation (PoC).

g. The optoacoustic system according to any of the aspects a) to f), wherein the processing unit is further configured to determine at least one distribution parameter (e.g. mean and/or maximal concentration) characterizing the spatial distribution of the concentration of the substance within a region of interest (ROI) of the concentration image.

h. The optoacoustic system according to aspect g), wherein the processing unit is further configured to provide i) the at least one distribution parameter and/or ii) a value (also referred to as “second value”) derived from the at least one distribution parameter and/or iii) diagnostic information derived from the at least one distribution parameter and/or from the value (“second value”) for further use.

i. The optoacoustic system according to any of the aspects a) to h) comprising a display unit configured to display information, wherein the data processing unit is further configured to control the display unit to display i) the at least one concentration image and/or ii) the at least one distribution parameter and/or iii) the value (“second value”) derived from the at least one distribution parameter and/or iv) the diagnostic information derived from the at least one distribution parameter and/or from the value (“second value”).

j. The optoacoustic system according to any of the aspects a) to i), wherein the irradiation unit is configured to irradiate the tissue with two or more wavelength sequences of electromagnetic radiation, wherein at each wavelength sequence the tissue is successively irradiated with electromagnetic radiation at the two or more different irradiation wavelengths (λₖ, k=1 . . . K), and the processing unit is configured
to correct at least one of the sequences of optoacoustic images (iₙ, n=1 . . . N) obtained for at least one of the different irradiation wavelengths (λₖ, k=1 . . . K) so as to at least partially compensate for a movement of the probe relative to the tissue while acoustic waves, which are successively generated in the tissue in response to successively irradiating the tissue with the electromagnetic radiation at the different irradiation wavelengths (λₖ, k=1 . . . K), were detected, and
to generate at least one of the compounded optoacoustic images (iₛ) from the at least one corrected sequence of optoacoustic images (iₙ, n=1 . . . N).

k. The optoacoustic system according to any of the aspects a) to j), wherein the processing unit is configured
to select, from the sequences of optoacoustic images (iₙ, n=1 . . . N) obtained for the different irradiation wavelengths (λₖ, k=1 . . . K), a sequence of optoacoustic images obtained for one of the different irradiation wavelengths (the images preferably comprising images of blood vessels), wherein images of the selected sequence of optoacoustic images exhibit a higher image contrast than images of the non-selected sequences of optoacoustic images obtained for the other irradiation wavelengths, and
to determine, based on the selected sequence of optoacoustic images, a trajectory of the probe (while the probe is being moved relative to the tissue).

I. The optoacoustic system according to aspect k), wherein the processing unit is configured
to correct at least one of the sequences of optoacoustic images (iₙ, n=1 . . . N) obtained for at least one of the different irradiation wavelengths (λₖ, k=1 . . . K) by considering the trajectory of the probe so as to at least partially compensate for the movement of the probe relative to the tissue while acoustic waves, which are successively generated in the tissue in response to successively irradiating the tissue with the electromagnetic radiation at the different irradiation wavelengths (λₖ, k=1 . . . K), were detected, and
to generate at least one of the compounded optoacoustic images (iₛ) from the at least one corrected sequence of optoacoustic images (iₙ, n=1 . . . N).

m. The optoacoustic system according to any of the aspects k) or I), wherein the processing unit is configured
to determine, based on the selected sequence of optoacoustic images, motion information, e.g. velocity and/or acceleration and/or rotation speed, regarding the movement of the probe.

n. The optoacoustic system according to any of the aspects k) to m), wherein the processing unit is configured
to control a display unit to display information regarding the trajectory and/or the motion information, e.g. velocity and/or acceleration and/or rotation speed, regarding the movement of the probe.

o. The optoacoustic system according to any of the aspects k) to n), wherein the processing unit is configured
to control a display unit to display instructional information (e.g. green light or “velocity of probe ok” or, respectively, red light or “velocity of probe too high”) classifying the movement of the probe (controlled and/or carried out by a user) based on a comparison of the motion information, e.g. velocity and/or acceleration and/or rotation speed, regarding the movement of the probe with predefined information, e.g. maximal velocity and/or acceleration and/or rotation speed, regarding the movement of the probe.

p. The optoacoustic system according to any of the aspects k) to o), wherein the processing unit is configured
to determine an overlap information regarding the extent of mutual overlap and/or correlation of the images of at least one of the sequences of optoacoustic images (iₙ, n=1 . . . N) obtained for at least one of the different irradiation wavelengths (λₖ, k=1 . . . K),
to control a display unit to display instructional information (e.g. green light or “velocity of probe ok” or, respectively, red light or “velocity of probe too high”) classifying the movement of the probe (controlled and/or carried out by a user) based on the overlap information.

x. An optoacoustic device comprising

an interface configured to receive optoacoustic data from a probe, in particular a handheld probe, the optoacoustic data relating to acoustic waves which were
generated in a tissue, in particular comprising muscle tissue, in response to an irradiation of the tissue with time-varying, in particular pulsed, electromagnetic radiation at two or more different irradiation wavelengths and
detected by a concave, in particular spherically shaped, matrix array of transducer elements of the probe while the probe is being moved, in particular translated and/or rotated, to a plurality of different probe positions, in particular including different locations and/or orientations of the probe, relative to the tissue, and a processing unit configured
to reconstruct, in particular 3D, optoacoustic images (also referred to as “single-wavelength optoacoustic images” or “single-wavelength MSOT images”) based on the optoacoustic data relating to the acoustic waves which were detected at the different irradiation wavelengths and probe positions so as to obtain, for each of the different irradiation wavelengths, a sequence of optoacoustic images (iₙ, n=1 . . . N), which were obtained for the different probe positions,
to generate, for each of the different irradiation wavelengths, a compounded, in particular 3D, optoacoustic image (iₛ) from the sequence of optoacoustic images (iₙ, n=1 . . . N) by

i) determining a location (Tₙ, n=2 . . . N) and/or rotation (θₙ, n=2 . . . N) of at least one optoacoustic image (iₙ) of the sequence relative to an initial optoacoustic image (i₁) of the sequence, and

ii) combining the at least one optoacoustic image (iₙ) of the sequence with the initial optoacoustic image (i₁) of the sequence so as to obtain the compounded optoacoustic image (iₛ) by considering the determined location (Tₙ) and/or rotation (θₙ) of the at least one optoacoustic image (iₙ) of the sequence relative to the initial optoacoustic image (i₁) of the sequence,
to determine at least one, in particular 3D, concentration image (also referred to as “MSP (multi-spectrally processed) image”) relating to a, in particular three-dimensional, spatial distribution of a concentration (also referred to as “first value”) of a substance, in particular a component and/or biomarker such as collagen, in the tissue based on the compounded optoacoustic images generated for the different irradiation wavelengths.

y. A method for analyzing optoacoustic data comprising the following steps:

s1) receiving optoacoustic data from a probe, in particular a handheld probe, wherein the optoacoustic data relate to acoustic waves which were
generated in a tissue, in particular comprising muscle tissue, in response to an irradiation of the tissue with time-varying, in particular pulsed, electromagnetic radiation at two or more different irradiation wavelengths and
detected by a concave, in particular spherically shaped, matrix array of transducer elements of the probe while the probe is being moved, in particular translated and/or rotated, to a plurality of different probe positions, in particular including different locations and/or orientations of the probe, relative to the tissue,

s2) reconstructing, in particular 3D, optoacoustic images (also referred to as “single-wavelength optoacoustic images” or “single-wavelength MSOT images”) based on the optoacoustic data relating to the acoustic waves which were detected at the different irradiation wavelengths and probe positions so as to obtain, for each of the different irradiation wavelengths, a sequence of optoacoustic images (iₙ, n=1 . . . N), which were obtained for the different probe positions, s3) generating, for each of the different irradiation wavelengths, a compounded, in particular 3D, optoacoustic image (iₛ) from the sequence of optoacoustic images (iₙ, n=1 . . . N) by

i) determining a location (Tₙ, n=2 . . . N) and/or rotation (θₙ, n=2 . . . N) of at least one optoacoustic image (iₙ) of the sequence relative to an initial optoacoustic image (i₁) of the sequence, and

ii) combining the at least one optoacoustic image (iₙ) of the sequence with the initial optoacoustic image (i₁) of the sequence so as to obtain the compounded optoacoustic image (iₛ) by considering the determined location (Tₙ) and/or rotation (θₙ) of the at least one optoacoustic image (iₙ) of the sequence relative to the initial optoacoustic image (i₁) of the sequence, and

s4) determining at least one, in particular 3D, concentration image (also referred to as “MSP (multi-spectrally processed) image”) relating to a, in particular three-dimensional, spatial distribution of a concentration (also referred to as “first value”) of a substance, in particular a component and/or biomarker such as collagen, in the tissue based on the compounded optoacoustic images generated for the different irradiation wavelengths.

z. A computer program product configured to cause a computer to execute the method according to the preceding aspect.

In the following, alternatives or further preferred embodiments of above aspects a) to z) are described:

1. Application of the location information of a wavelength (with ideal contrast, e.g. blood vessels) to all acquired wavelengths to obtain a multispectral set. Here it is possible to also include a plausibility check by e.g. correlation of overlapping image/volume ranges after rotation/translation.

2. Using the location information in recordings as a live motion indication. This information can be displayed to a user as a coordinate system, i.e. a kind of integrated navigation. This can, for example, also make it easier to keep still during kinetic recordings.

3. The calculation of a live confidence metric that is displayed to the user as a traffic light (e.g. red if the movement is too fast and the overlap is insufficient) to avoid incorrect stitching and thus false diagnoses.

4. In addition, it is possible or preferred to interpolate the trajectory in 3D vector space, since one can assume quasi-continuous movements. Thus, one can then draw conclusions about the images of the other wavelengths and if necessary
output a user warning, e.g. similarly to a traffic light
rotate images according to the curve instead of relying on “native” stitching for images with poor contrast. The plausibility check described in 1.) above can also be applied here.

5. Preferably, overlapping images can be used after rotation and translation to improve the contrast/SNR using correlation.

6. Alternatively, a delay-average-and-sum approach is used—in present case, preferably a translate/rotate-average-sum approach, where the main focus is on average. Especially in the image border areas (here the noise is high, because the tomographic coverage is poor), it is possible to achieve significantly higher contrast. Also the average resolution is even higher there.

7. Further, a two-part process is preferred, in which first the location coordinates are determined live by means of fast reconstruction, and then a complete model is reconstructed in high resolution (model based reconstruction). Although there may be memory limitations, between 5 and 100 recording positions work with an appropriate hardware to achieve the desired results, which would have immense advantages, since the tomographic effect of the acquisitions is fully exploited—e.g., instead of adding up image noise in the peripheral areas.

8. The approach described in 7.) can also be regarded as a kind of “moving average” approach to reduce the memory requirements, i.e. to reconstruct only 5-10 adjacent images together to intermediate volumes, and then to stitch them again.

9. Preferably, the above aspects can be applied also to other chromophores such as lipids.

10. Preferably, 3D visualization is achieved by rendering using e.g. shear warp method, maximum intensity projections or the like.

11. Alternatively, regarding the detector shape a 3D shape with a “given direction of movement” (rather than a spherical shape) may be preferred, e.g. in the form of a “cut open barrel”, i.e. a concave half cylinder-shaped matrix array) instead of a half sphere. This way, the resolution perpendicular to the direction of movement (parallel to the cylinder axis) remains high (as with the spherical detectors), but is reduced in the direction of movement (parallel to the cylinder axis). However, this increases the field of view in the direction of movement (parallel to the cylinder axis), thus increasing the overlap of the individual volumes. This increases the confidence in stitching and, due to the higher overlap, the resolution can be compensated by tomography (see 7.) and 8.) above).

12. The large imaging volumes obtained with the embodiments according to the above aspects are particularly advantageous in connection with diagnostic applications, as they allow for a macroscopic view that is otherwise only possible with whole-body imaging. Also see visualization above, but specifically, one could again elaborate the statistical evaluation of the large area, which could be dilated, eroded, opened and closed and thus segmented. Especially for the chromophore channels, where in DMD the collagen can be evaluated as a flat or areal effect. On such a segmented image (possibly by means of a threshold value in the collagen channel) one can then calculate and analyse area or area components, or signal variation (STD, STDERR).

Alternatively or additionally, the objects and/or advantages disclosed herein may be preferably achieved by a device, an optoacoustic system, a computer program and methods according to at least one of the following aspects A to S.

A. A device for analyzing optoacoustic data comprising:
An interface for receiving optoacoustic data from a detection unit of an optoacoustic imaging system, wherein the optoacoustic data relate to acoustic waves that are generated in a tissue in response to irradiation of the tissue with time-varying electromagnetic radiation at two or more different irradiation wavelengths (λ), wherein the tissue comprises at least one of a muscle tissue, connective tissue, organ, tendon and/or pathogenic (fibrotic) tissue and
A processor to analyze the optoacoustic data to (i) determine a spatial distribution of at least one first value of an optoacoustic collagen signal (in a.u.), which relates to a concentration of collagen in the tissue, based on the optoacoustic data, (ii) derive at least one second value (e.g. collagen_mean, collagen_max, collagen_mean/collagen_max) which corresponds to or is derived from at least one distribution parameter (e.g. mean/max) characterizing the spatial distribution of the at least one first value of an optoacoustic collagen signal within a region of interest (ROI) of the spatial distribution of the at least one first value of an optoacoustic collagen signal, and (iii) provide the at least one second value and/or diagnostic information derived from the at least one second value for further use to a display unit for display.

B. The device according to aspect A, wherein the spatial distribution of the at least one first value of an optoacoustic collagen signal is two-dimensional or three-dimensional.

C. The device according to aspect A or B, wherein the at least one second value corresponds to or is derived from at least one of the following distribution parameters:
a mean value of the spatial distribution of the at least one first value of an optoacoustic collagen signal within the region of interest, and/or
a maximum value of the spatial distribution of the at least one first value of an optoacoustic collagen signal within the region of interest.

D. The device according to aspect C, wherein the at least one second value is derived from the mean value and the maximum value of the spatial distribution of the at least one first value of an optoacoustic collagen signal within the region of interest.

E. The device according to aspect C or D, wherein the at least one second value corresponds to a ratio between the mean value and the maximum value of the spatial distribution of the at least one first value of an optoacoustic collagen signal within the region of interest.

F. The device according to any of the preceding aspects, wherein the processor is further configured to derive the diagnostic information from the at least one second value by comparing the at least one second value with at least one predefined reference value.

G. The device according to any of the preceding aspects, wherein the derived diagnostic information relates to the presence or absence and/or likelihood of the presence or absence of a muscle disorder, in particular Duchenne muscular dystrophy (DMD), in the tissue.

H. The device according to any of the preceding aspects, wherein the processor is further configured to
reconstruct an ultrasound image of the tissue based on ultrasound data (detector signals) relating to ultrasound waves reflected by the tissue in response to ultrasound waves impinging on the tissue, and
provide the ultrasound image of the tissue for displaying the ultrasound image of the tissue on a display unit.

I. The device according to any of the preceding aspects, wherein the a display unit is configured to display information, and wherein the processor is further configured to control the display unit to display
the spatial distribution of the at least one first value of an optoacoustic collagen signal, which relates to a concentration of collagen in the tissue, and/or
the ultrasound image of the tissue and/or
the at least one second value and/or’
the diagnostic information derived from the at least one second value.

J. The device according to aspect I, wherein the processor is further configured to merge the spatial distribution of the at least one first value of an optoacoustic collagen signal, which relates to a concentration of collagen in the tissue, and the ultrasound image of the tissue to obtain a merged optoacoustic-ultrasound image of the tissue, and to control the display unit to display the merged optoacoustic-ultrasound image of the tissue.

K. The device according to aspect I or J, wherein the display unit and/or the processor is further configured to enable a user to select the region of interest (ROI), within which the at least one second value is derived from the spatial distribution of the at least one first value, in the displayed spatial distribution of the at least one first value and/or in the displayed merged optoacoustic-ultrasound image of the tissue.

L. An optoacoustic system for generating and analyzing optoacoustic data, the system comprising
an irradiation unit configured to irradiate a tissue comprising muscle tissue with electromagnetic radiation at two or more different irradiation wavelengths (λ), said electromagnetic radiation having a time-varying, in particular pulsed, intensity,
a detection unit configured to detect acoustic waves generated in the tissue in response to irradiating the tissue with the electromagnetic radiation at the different irradiation wavelengths (λ) and to generate according optoacoustic data, and
the device for analyzing optoacoustic data according to any preceding aspect.

M. The system according to aspect L, wherein the irradiation unit is configured to irradiate the tissue with electromagnetic radiation at two or more different irradiation wavelengths (λ) being in a wavelength range between 650 nm and 1200 nm, preferably between 680 nm and 1100 nm.

N. A method for analyzing optoacoustic data, the method comprising the following steps:
receiving optoacoustic data (detector signals) relating to acoustic waves generated in the tissue in response to irradiating the tissue with time-varying electromagnetic radiation at two or more different irradiation wavelengths (λ)
determining a spatial distribution of at least one first value (optoacoustic collagen signal in a.u.), which relates to a concentration of collagen in tissue comprising at least one of a muscle tissue, connective tissue, organ, tendon and/or pathogenic (fibrotic) tissue, and is based on the optoacoustic data,
deriving at least one second value (e.g. collagen_mean, collagen_max, collagen_mean/collagen_max) from the spatial distribution of the at least one first value, the at least one second value corresponding to or being derived from at least one distribution parameter (e.g. mean/max) characterizing the spatial distribution of the at least one first value within a region of interest of the spatial distribution of the at least one first value, and
providing the at least one second value and/or diagnostic information derived from the at least one second value to a display unit for display.

O. A computer program product which causes a computer to execute the method according to the previous aspect.

P. A method of diagnosing a muscle disorder, in particular Duchenne muscular dystrophy (DMD), in a patient, comprising:
irradiating a tissue in the patient with time-varying electromagnetic radiation at two or more different irradiation wavelengths (λ), wherein the tissue comprises at least one of a muscle tissue, connective tissue, organ, tendon and/or pathogenic (fibrotic) tissue,
determining a spatial distribution of at least one optoacoustic collagen signal (e.g. in arbitrary units a.u.), which relates to a concentration of collagen in the tissue based on optoacoustic data (detector signals) relating to acoustic waves generated in the tissue in response to irradiating the tissue,
deriving at least one second value (e.g. either collagen_mean, collagen_max, and/or collagen_mean/collagen_max), which corresponds to or is derived from at least one distribution parameter characterizing the spatial distribution of the at least one optoacoustic collagen signal within a region of interest of the spatial distribution of the at least one optoacoustic collagen signal, and
outputting, in particular displaying on a display unit, the at least one second value and/or diagnostic information which has been derived from the at least one second value by comparing the at least one second value with at least one predefined reference value and which relates to the presence or absence and/or likelihood of the presence or absence of a muscle disorder, in particular Duchenne muscular dystrophy (DMD), in the tissue of the patient.

Q. A method of treating a muscle disorder in a patient, in particular Duchenne muscular dystrophy (DMD), comprising:
irradiating a tissue in the patient with time-varying electromagnetic radiation at two or more different irradiation wavelengths (λ), wherein the tissue comprises at least one of a muscle tissue, connective tissue, organ, tendon and/or pathogenic (fibrotic) tissue,
determining a spatial distribution of at least one optoacoustic collagen signal (e.g. in arbitrary units a.u.), which relates to a concentration of collagen in the tissue based on optoacoustic data (detector signals) relating to acoustic waves generated in the tissue in response to irradiating the tissue,
deriving at least one second value (e.g. either collagen_mean, collagen_max, collagen_mean/collagen_max), which corresponds to or is derived from at least one distribution parameter characterizing the spatial distribution of the at least one optoacoustic collagen signal within a region of interest of the spatial distribution of the at least one optoacoustic collagen signal, and
outputting, in particular displaying on a display unit, the at least one second value and/or diagnostic information which has been derived from the at least one second value by comparing the at least one second value with at least one predefined reference value and which relates to the presence or absence and/or likelihood of presence or absence of a muscle disorder, in particular Duchenne muscular dystrophy (DMD), in the tissue of the subject, and
treating the subject, preferably by administering a medication to the subject, in accordance with the diagnostic information.

R. A method of treatment as described in aspect Q, wherein the course of treatment depends on but is not limited to:
selecting the medication and/or an active agent of the medication depending on the at least one second value and/or diagnostic information,
selecting the dose of an active agent contained in the medication depending on the at least one second value and/or diagnostic information,
selecting time and/or duration of administering the medication depending on the at least one second value and/or diagnostic information.

S. A method of treatment as described in aspect R, wherein treating the subject with a medication comprises:
administering at least one corticosteroid, such as Deflazacort and/or Ataluren (Translarna™, PTC Therapeutics Inc.) and/or Spinraza® (Nusinersen) and/or Zolgensma® (Onasemnogene abeparvovec) or other approved drugs, and/or
administering one or more of Eteplirsen (Exondys 51®), corticosteroids, such as prednisone, and/or heart medications, such as angiotensin-converting enzyme (ACE) inhibitors or beta blockers.

The above and other elements, features, characteristics, advantages and/or alternatives of present invention will become more apparent from the following figures and appendixes showing and/or describing preferred embodiments of aspects of the invention:

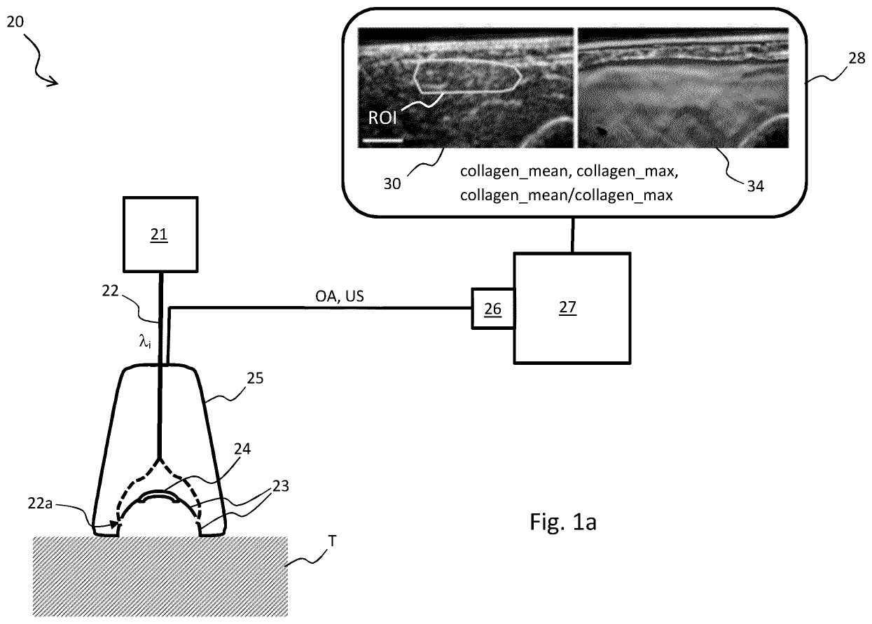

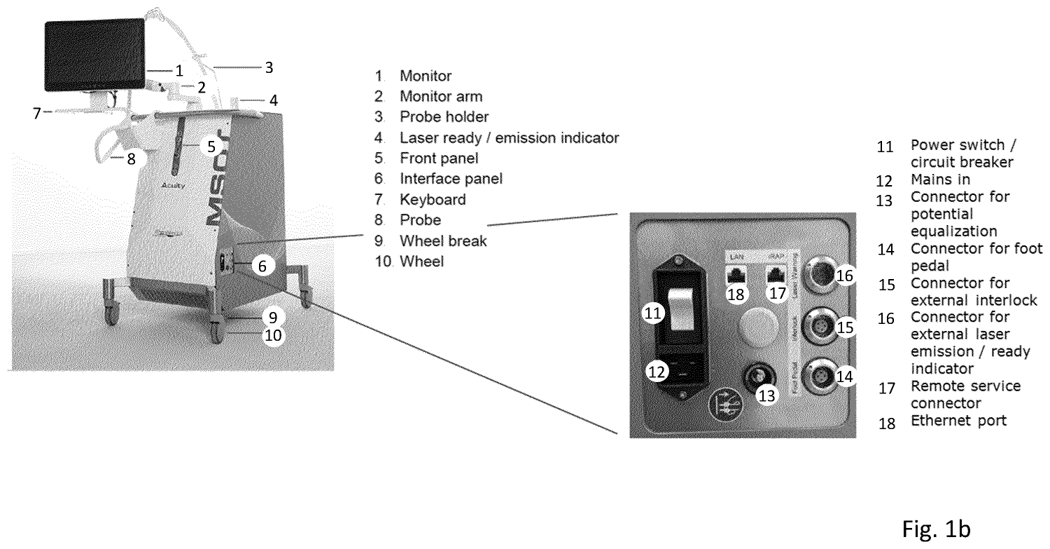

FIG. 1 a) an example of an optoacoustic system and b) an example of a system “MSOT Acuity”;

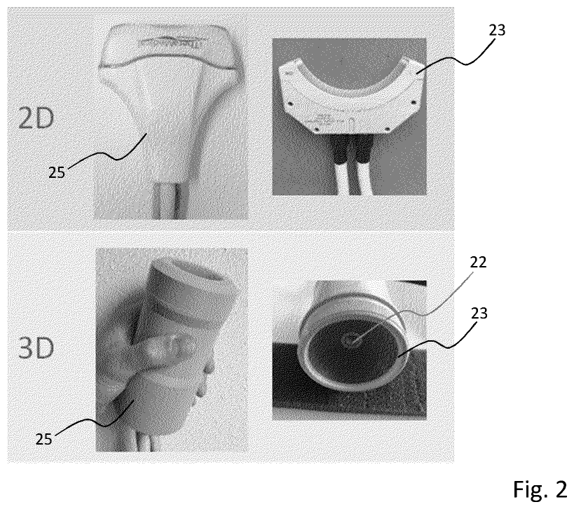

FIG. 2 examples of preferred detectors for acquiring 2D and 3D optoacoustic images;

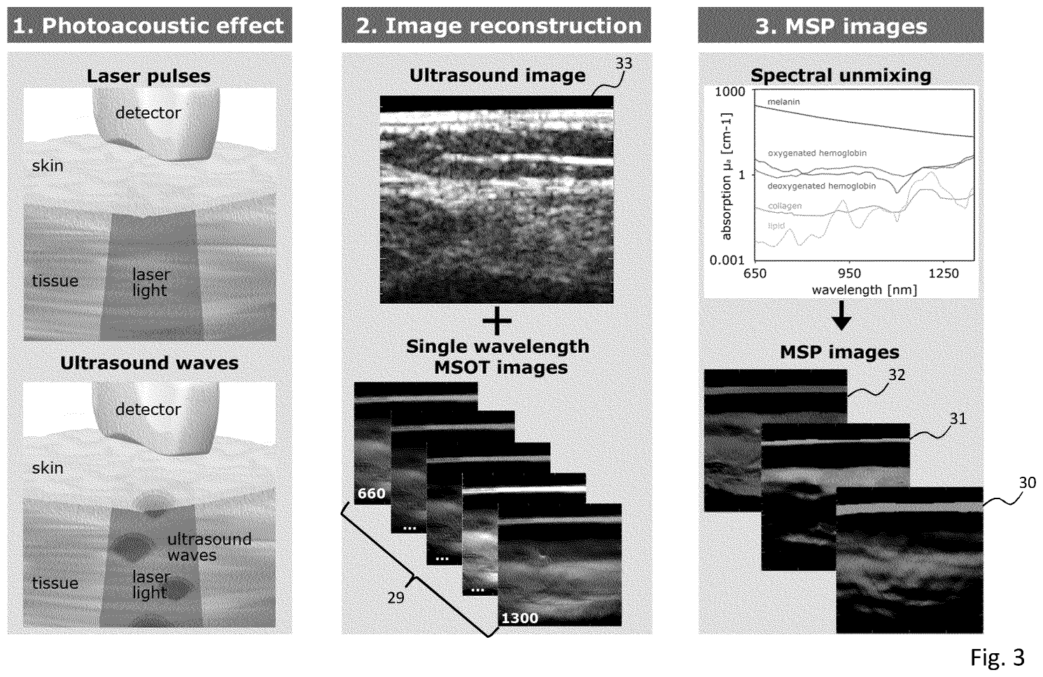

FIG. 3 a diagram illustrating preferred steps of a method;

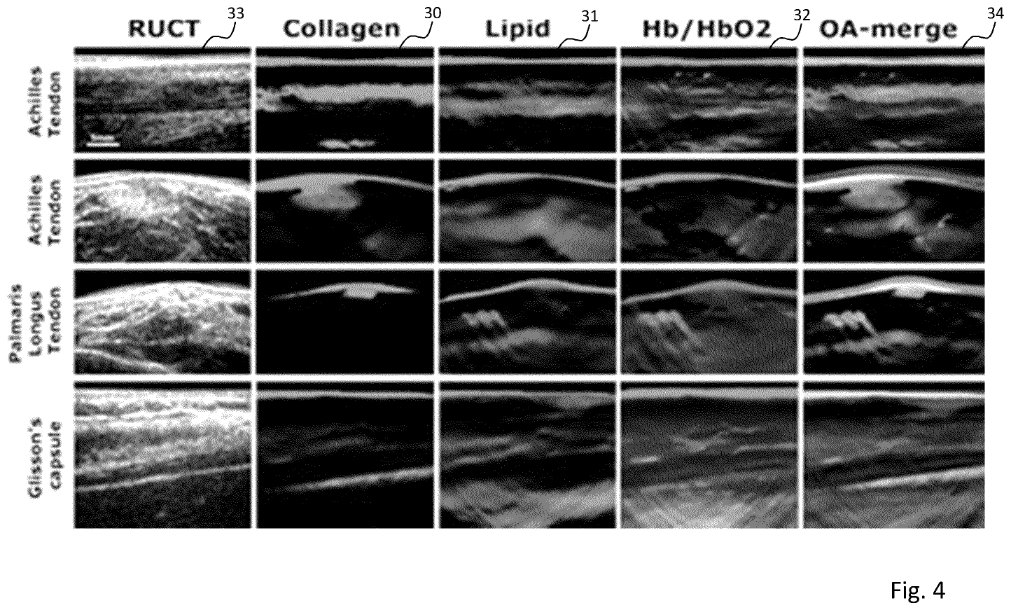

FIG. 4 a diagram showing examples of images of different tissues;

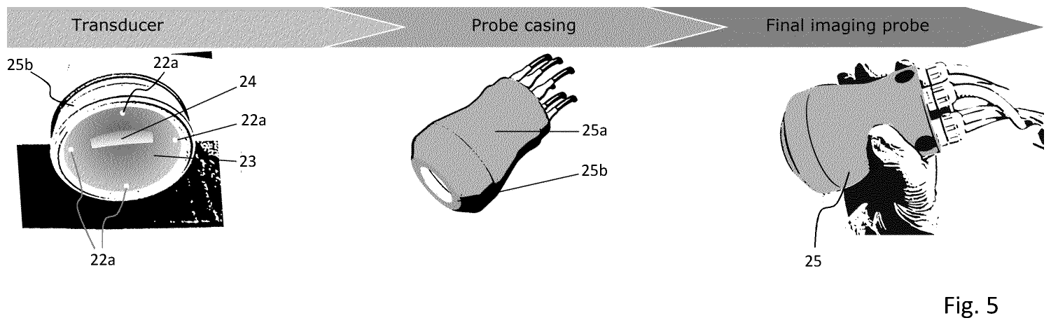

FIG. 5 another example of a preferred detection unit;

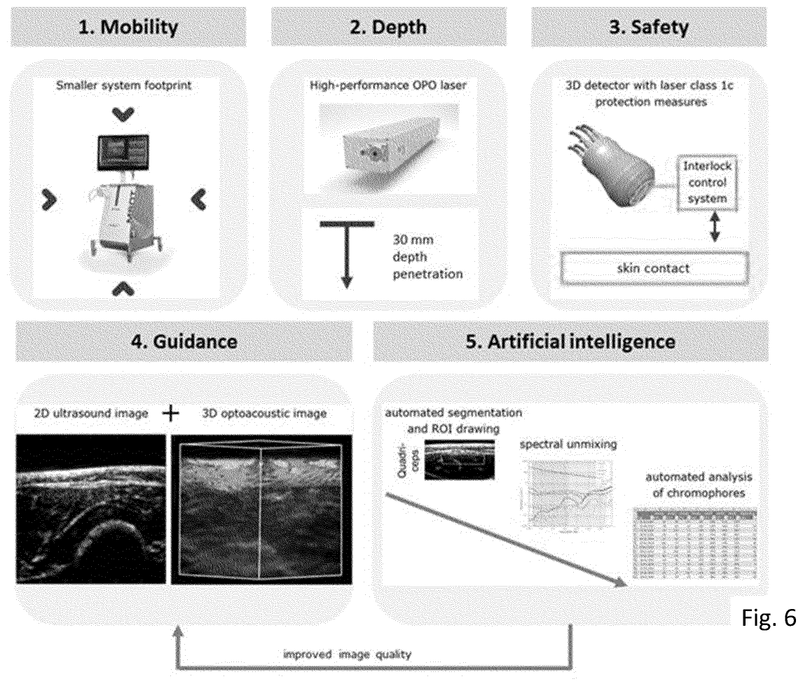

FIG. 6 a diagram illustrating an example of an application of the preferred detection unit;

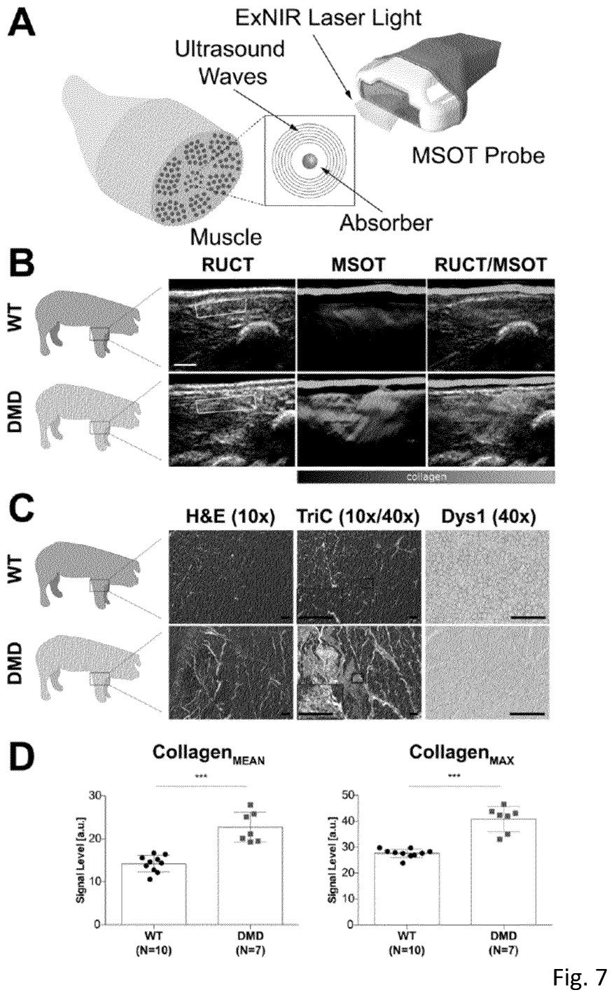

FIG. 7 a diagram illustrating in vivo 2D MSOT imaging of newborn piglets;

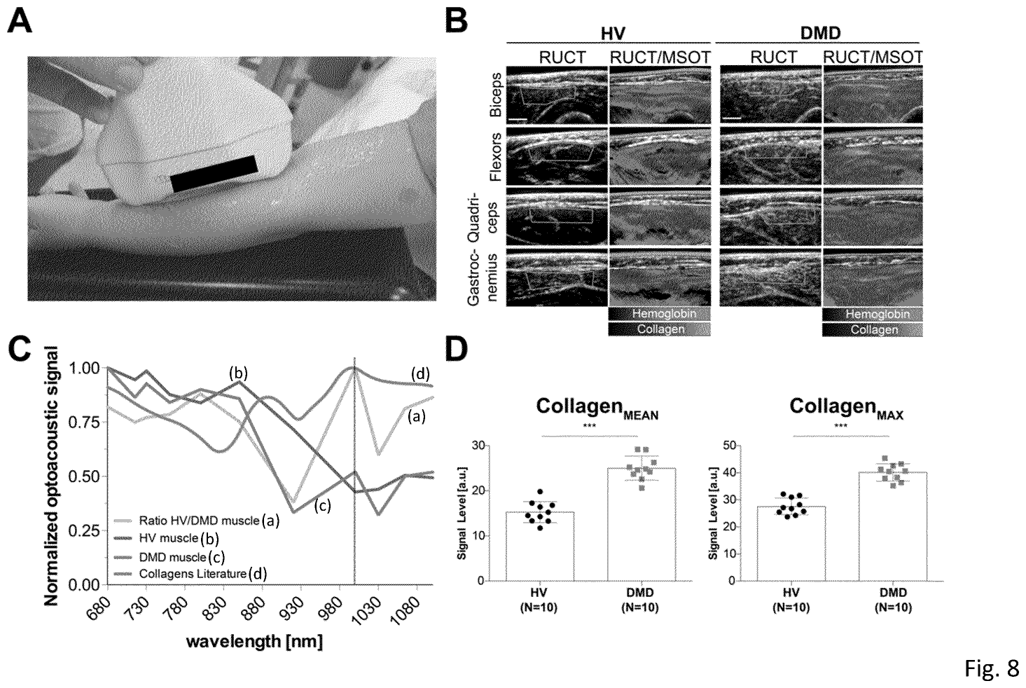

FIG. 8 a diagram illustrating in vivo 2D MSOT imaging and collagen quantification in DMD patients and healthy volunteers (HV);

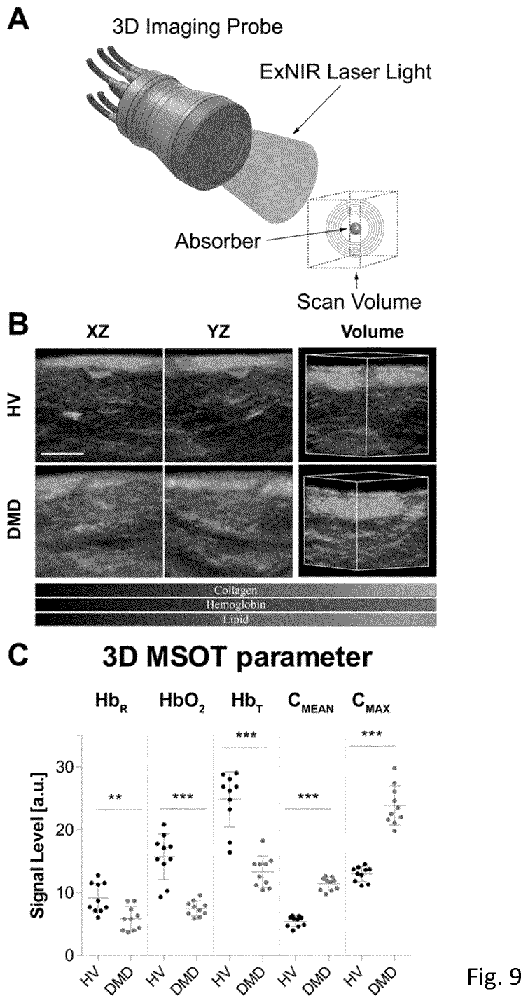

FIG. 9 a diagram illustrating in vivo 3D MSOT imaging in DMD patients and healthy volunteers;

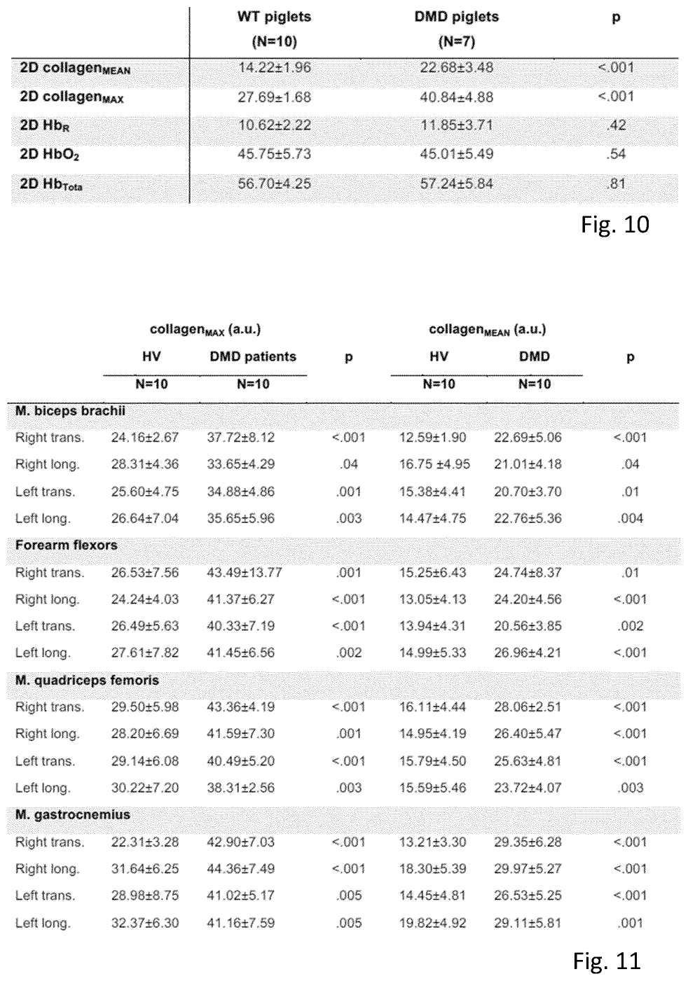

FIG. 10 a table giving an overview of piglet 2D-MSOT parameters:

FIG. 11 a table giving an overview of mean and maximum 2D-MSOT collagen signal sorted by scanning region;

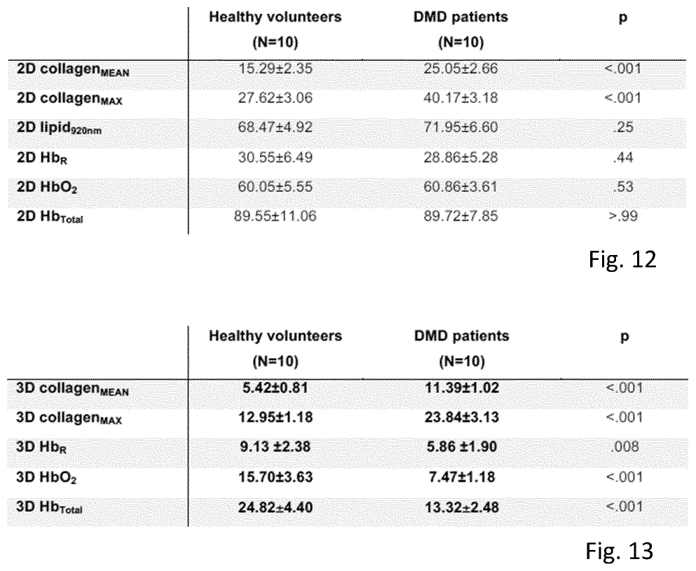

FIG. 12 a table giving an overview of human 2D-MSOT parameters;

FIG. 13 a table giving overview of human 3D-MSOT parameters;

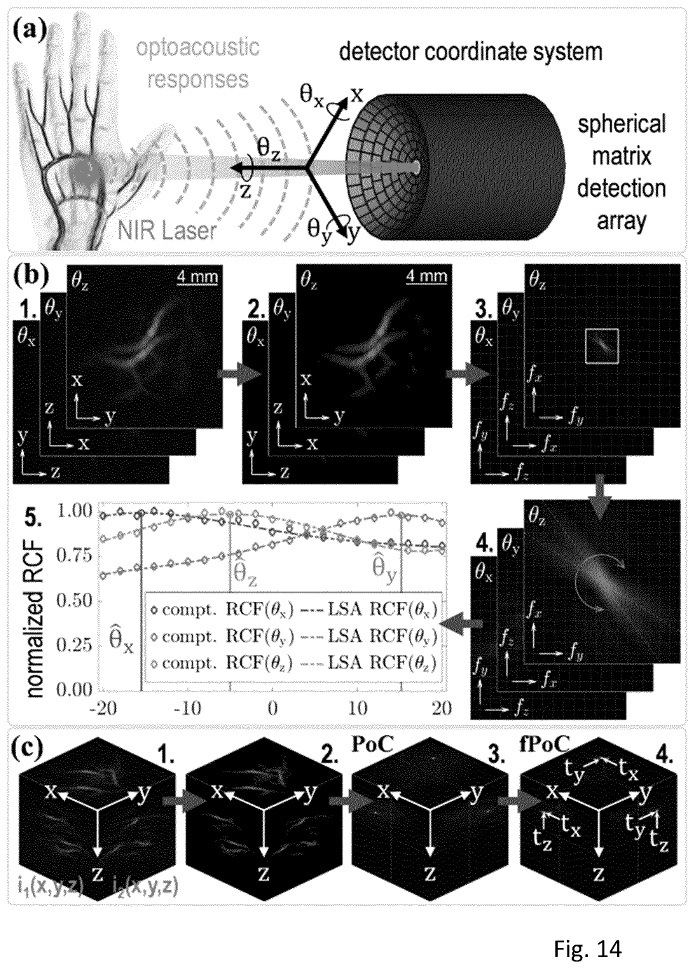

FIG. 14 a schematic diagram illustrating an example of Fourier-based 3D motion estimation;

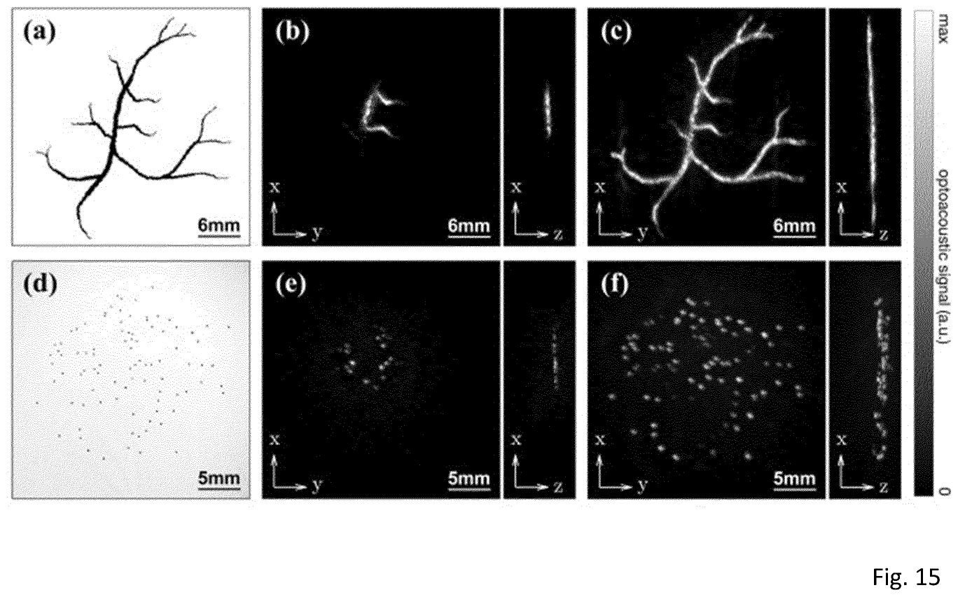

FIG. 15 a schematic diagram illustrating examples of spatial image compounding results for phantom scans;

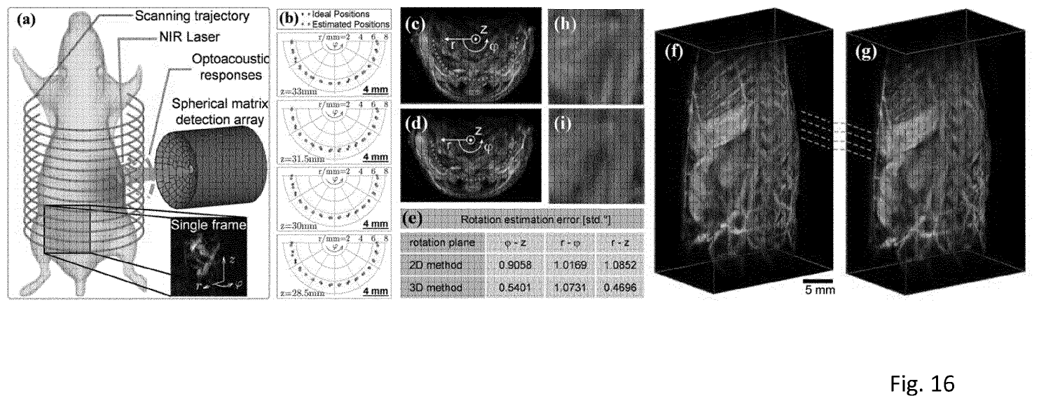

FIG. 16 a schematic diagram illustrating an example of Fourier-based spatial compounding for a spiral volumetric optoacoustic tomography (SVOT) scan;

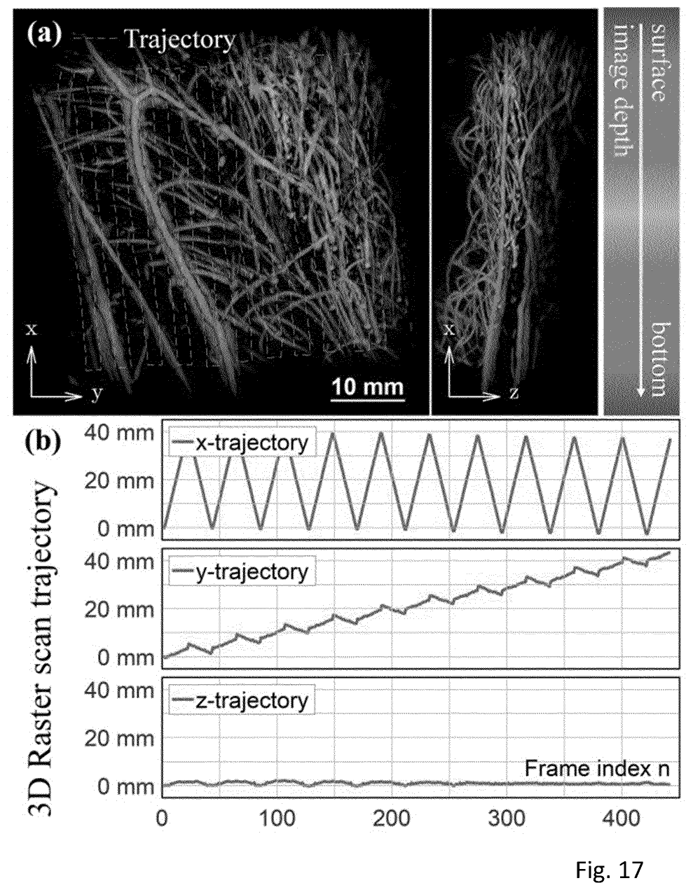

FIG. 17 a schematic diagram illustrating exemplary results of volumetric optoacoustic angiography of a human arm performed in a zigzag-scan pattern; and

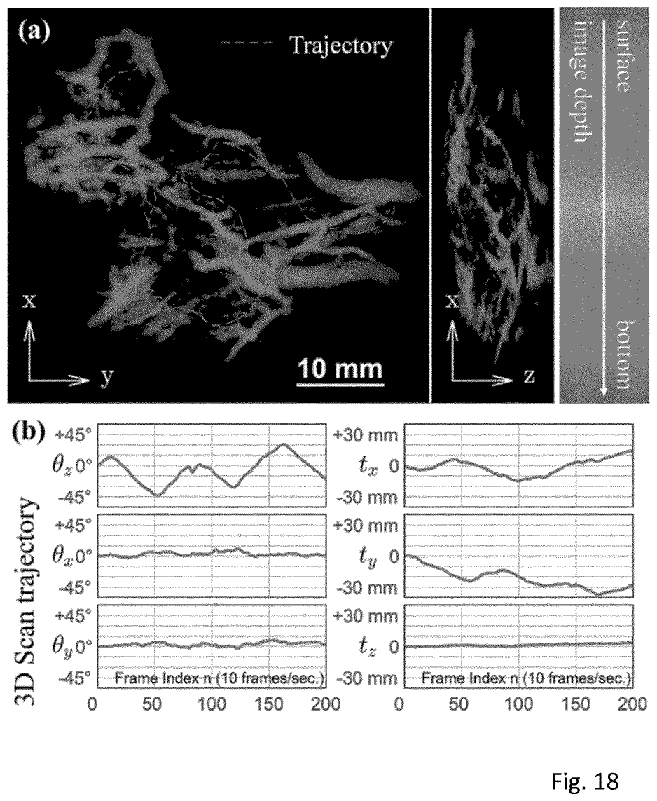

FIG. 18 a schematic diagram illustrating exemplary results of freehand optoacoustic human angiography of a human palm along an arbitrary trajectory.

FIG. 1a shows a schematic representation of an example of a system 20 for generating and analyzing optoacoustic data. The system 20 comprises an irradiation unit 21, 22 configured to irradiate a tissue T, in particular muscle tissue, with electromagnetic radiation at two or more different irradiation wavelengths λᵢ, said electromagnetic radiation having a time-varying, in particular pulsed, intensity.

The irradiation unit 21, 22 preferably comprises a radiation source 21, e.g. a wavelength-tunable laser like an OPO laser, generating the electromagnetic radiation at the two or more different irradiation wavelengths and a guiding element 22, e.g. optical fiber(s) or an optical fiber bundle, configured to guide the electromagnetic radiation generated by the radiation source 21 to the tissue T.

Further, the system 20 comprises a first detection unit 23 configured to detect acoustic waves, also referred to as optoacoustic waves, generated in the tissue T in response to irradiating the tissue T with the electromagnetic radiation at the different irradiation wavelengths λᵢ and to generate according optoacoustic signals OA based on which optoacoustic images can be reconstructed.

Preferably, the detection unit 23 comprises a plurality of, e.g. 256 or 512 or more, first ultrasound transducers arranged on a two-dimensional concave surface and configured and/or controlled to detect the optoacoustic waves. Preferably, the first ultrasound transducers form a spherically shaped matrix array.

Optionally, a second detection unit 24 can be provided, which is configured to transmit ultrasound waves towards the tissue T and to detect ultrasound waves reflected by the tissue T and to convert same to according ultrasound signals US based on which ultrasound images, also referred to as reflection ultrasound computed tomography (RUCT) images, can be reconstructed.

Preferably, the second detection unit 24 comprises an array, preferably a curved or straight linear array, of second ultrasound transducers which are arranged in a region at or near the apex of the concave surface and configured and/or controlled to both transmit and detect ultrasound waves.

In present example, a distal end of the guiding element 22 including one or more distal end sections (dashed), the first detection unit 23 and the (optional) second detection unit 24 are integrated in a handheld probe 25.

In present example, the distal end sections (dashed) of optical fibers of the guiding element 22 are provided in illumination openings 22a which are provided in a circumferential region of the spherical matrix array of the first detection unit 23. Alternatively or additionally, a distal end of the guiding element 22 and/or the distal end sections (dashed) of optical fibers of the guiding element 22 is or, respectively, are located at the or in a region at the apex of the concave matrix array of the first detection unit 23.

As exemplarily shown in FIG. 2 (top), when the probe 25 is configured to generate optoacoustic signals for 2D optoacoustic images, the first detection unit 23 preferably comprises an arc-shaped (concave) linear array of transducer elements.

As exemplarily shown in FIG. 2 (bottom), when the probe 25 is configured to generate optoacoustic signals for 3D optoacoustic images, the first detection unit 23 preferably comprises a spherically shaped (concave) matrix array of transducer elements, wherein a distal end of the guiding element 22 is located at the apex of the concave matrix array.

As further shown in FIG. 1a, optoacoustic signals OA generated by the first detection unit 23 and, optionally, ultrasound signals US generated by the second detection unit 24 are guided, preferably via an optional interface 26, to a data processor 27, e.g. a computer system, which is configured to process and/or analyze the optoacoustic signals OA and/or ultrasound signals US.

Preferably, the data processor 27 is configured to reconstruct optoacoustic images, also referred to as single-wavelength MSOT images, based on the optoacoustic signals OA obtained at the different irradiation wavelengths λᵢ.

Algorithms for reconstructing optoacoustic images based on optoacoustic signals are known in the art. For example, a preferably used back-projection algorithm is described by Xu and Wang, “Universal back-projection algorithm for photoacoustic computed tomography”, Phys. Rev. E 71, 016706, 19 Jan. 2005, which is incorporated by reference herewith.

Within the meaning of present disclosure, the term “optoacoustic data” preferably relates to optoacoustic images, in particular single-wavelength MSOT images 29 (see FIG. 3 and respective description below), which are reconstructed based on optoacoustic signals OA obtained at different irradiation wavelengths λᵢ and/or to the optoacoustic signals OA obtained at the different irradiation wavelengths λᵢ.

Preferably, the data processor 27 is configured to determine a spatial distribution of at least one first value relating to a concentration of collagen in the tissue T based on the optoacoustic data, preferably based on optoacoustic images 29 (see FIG. 3) obtained at different irradiation wavelengths.

Within present disclosure, the term “first value relating to a concentration of collagen in the tissue” is also referred to as “collagen signal”, which is preferably given in arbitrary units (a.u.). Further, the term “spatial distribution” with respect to the at least one first value preferably relates to a 2D or 3D image, also referred to as an MSP (multi-spectrally processed) image 30 (see FIG. 3), showing the concentration of collagen in the tissue and/or the collagen signal.

The data processor 27 is further configured to derive at least one second value from the spatial distribution of the at least one first value, wherein the at least one second value corresponds to or is derived from at least one distribution parameter, e.g. a mean and/or maximum value, characterizing the spatial distribution of the at least one first value within a region of interest (ROI) of the spatial distribution of the at least one first value.

Preferably, the at least one second value corresponds to a mean value, also referred to as “collagen_mean”, and/or a maximum value, also referred to as “collagen_max”, of the collagen signal or concentration of collagen within an ROI in the 2D or 3D image, in particular the MSP image, showing the spatial distribution of collagen in the tissue.

For example, the mean value can be an average value, e.g. an arithmetic mean value, or a median value of the collagen signal values within the ROI.

Alternatively or additionally, the at least one second value is derived from the mean value, also referred to as “collagen_mean”, and/or the maximum value, also referred to as “collagen_max”, of the collagen signal or concentration of collagen within the region of interest in the 2D or 3D image, in particular the MSP image, e.g., by dividing collagen_mean/collagen_max.

Further, the data processor 27 is configured to output the at least one second value, e.g. collagen_mean, collagen_max and/or collagen_mean/collagen_max, and/or diagnostic information derived from the at least one second value for further use, e.g. for diagnostic purposes, in particular by displaying the at least one second value and/or diagnostic information on a display unit 28, e.g. a computer monitor.

In present example, the display unit 28 displays an ultrasound image 33, preferably a RUCT image, of the tissue T and a merged image 34, which is obtained by merging the ultrasound image 33 with at least one optoacoustic image, in particular MSP image 30 (see FIG. 3), of the tissue T showing the spatial distribution of at least collagen in the tissue T.

Preferably, the region of interest ROI of the MSP image, within which a mean and/or maximum value of the collagen concentration is determined, is preferably determined and/or selected by means of and/or based on and/or in the RUCT image 33. In this way, the investigator is anatomically guided, in particular to advantageously ensure that the at least one second value, in particular collagen_mean, collagen_max and/or collagen_mean/collagen_max, is derived for the desired region of interest of the tissue, e.g. from muscle tissue rather than skin tissue.

Preferably, the data processor 27 is configured to control the display unit 28 and/or a selection unit (not shown) such that the investigator can determine and/or select the ROI. Preferably, the ROI has a polygonal shape.

In present example, the display unit 28 further displays the at least one second value, preferably collagen_mean, collagen_max and/or collagen_mean/collagen_max, from which an investigator may derive diagnostic information and/or conclusions relating to the presence or absence and/or likelihood of presence or absence of a muscle disorder, in particular Duchenne muscular dystrophy. Alternatively or additionally, the data processor 27 may be configured to derive diagnostic information and/or conclusions relating to the presence or absence and/or likelihood of presence or absence of a muscle disorder, in particular Duchenne muscular dystrophy, and to display same at the display unit 28.

FIG. 1b shows a perspective view (left) of an example of a system “MSOT Acuity”, and an enlarged view (right) of an interface panel 6 located at a side area of the system. Preferably, the system comprises two detectors (e.g. a first detection unit 23 and a second detection unit 24, see FIG. 1a) and is configured for optoacoustic and ultrasound imaging of biological tissue and for generating and displaying hybrid optoacoustic-ultrasound images, in particular by merging the spatial distribution of at least one first value, which relates to a concentration of collagen in the tissue, and the ultrasound image of the tissue to obtain a merged optoacoustic-ultrasound image of the tissue, and to control the monitor 1 to display the merged optoacoustic-ultrasound image of the tissue. Preferably, the system shown in FIG. 1b comprises at least a part of the components described above with reference to FIG. 1a. In particular, probe 8 and monitor 1 shown in FIG. 1b correspond to the probe 25 and display unit 28, respectively, described above with reference to FIG. 1a.

FIG. 3 shows a diagram illustrating preferred steps of a method as follows:

1. Photoacoustic effect: Tissue, in particular muscle tissue below the skin, is irradiated with pulsed laser light. Ultrasound waves, which are generated in response to absorption of pulsed laser light by the tissue, are detected by a detector.

2. Image reconstruction: Based on the ultrasound waves detected in response to irradiating the tissue with pulsed laser light at, in present example five, different wavelengths between 660 and 1300 nm, single-wavelength MSOT images 29 at the different wavelengths are reconstructed. Further, based on ultrasound waves transmitted towards to and reflected by the tissue an ultrasound image(s) 33 is (are) reconstructed.

3. MSP (multi-spectrally processed) images: Based on the reconstructed single-wavelength MSOT images 29 at the different wavelengths and (known) absorption coefficients μₐ of one or more biomarkers at the different wavelengths, one or more MSP images 30 to 32 are derived, preferably by means of so-called spectral un-mixing, wherein each of the MSP images 30 to 32 represents a spatial distribution regarding a concentration of at least one biomarker in the tissue. In present example, the diagram shows the dependence of the absorption coefficient μₐ of melanin, oxygenated hemoglobin, deoxygenated hemoglobin, collagen and lipid from the wavelength in a spectral region comprising the five irradiation wavelengths (between 660 and 1300 nm) at which MSOT images were obtained. In present example, a first MSP image 30 relates to a concentration of collagen, a second MSP image 31 relates to a concentration of lipid and a third MSP image 32 relates to a concentration of oxygenated and deoxygenated hemoglobin in the tissue. The first MSP image 30 corresponds to a “spatial distribution of at least one first value which relates to a concentration of collagen in a tissue” according to an aspect of present disclosure.

In general, the contrast of MSOT imaging is due to photo-absorbing moieties that can be intrinsic to tissue, for example collagen, hemoglobin, melanin and/or lipid or expressed molecules (e.g. fluorescent proteins), or extrinsically administered such as fluorescent agents or nanoparticles. Despite the generally high resolution of MSOT imaging, this is typically not high enough to resolve substances in a molecular level. For this reason, each MSOT pixel (voxel), corresponds usually to more than one photo-absorber and has a spectral response that is a linear combination of the spectral responses of all these absorbers. Due to this per pixel spectral mix, an MSOT pixel can be referred to as a “mixed pixel”, and the molecular targets of interest can be referred to as “sub-pixel” targets. Since the targets typically lie in a subpixel level, so-called spectral un-mixing methods can produce robust solutions for this problem. Spectral un-mixing is then a process of estimating the discrete material components with distinctive spectral signatures (e.g. absorption coefficients μₐ of one or more components/biomarkers at different wavelengths) from multispectral measurements (e.g. single-wavelength MSOT images 29).

Accordingly, optoacoustic imaging of different moieties, such as collagen, hemoglobin, melanin and/or lipid, of a tissue preferably comprises resolving their distinct absorption spectra. This is preferably achieved by illuminating the imaged tissue T at multiple wavelengths and performing spectral un-mixing operations in the collected optoacoustic data (single-wavelength MSOT images 29). In this way, MSP (multi-spectrally processed) images 30 to 32 of high spatial and temporal resolution are obtained, that are related to tissue optical absorption of biomarkers and, therefore, to the concentration of the respective biomarker in the tissue T.

Preferably, spectral un-mixing of the collected optoacoustic data is performed by applying at least one of the algorithmic approaches described by Tzoumas et al., “Un-mixing Molecular Agents From Absorbing Tissue in Multispectral Optoacoustic Tomography”, IEEE Transactions on Medical Imaging, Volume 33, Issue: 1, January 2014, pages 48-60, which is incorporated by reference herewith.

FIG. 4 shows examples of images of different tissues obtained with reflection ultrasound computed tomography (RUCT) and optoacoustic imaging revealing spatial distributions of collagen, lipid and Hb/HbO2 concentration as well as merged images 34 (“OA-merge”) each including both a RUCT image 33 and the optoacoustic images 30 to 32 of the respective tissue.

FIG. 5 shows perspective views of an example of a detection unit, also referred to as handheld probe 25, which is preferably used with or in the device, system and/or methods according to preferred aspects of the invention.

Handheld probe 25 comprises a probe casing section 25a and a sensor section 25b, in which both a first detection unit 23 (spherical matrix array) and a second detection unit 24 (linear array segment) are integrated. Further, illumination openings 22a are provided at a circumferential region of the spherical matrix array of the first detection unit 23. For example, end sections of optical fibers of the guiding element 22 (see FIG. 1 a) are provided in the illumination openings 22a.

The shown example relates to a novel transducer which combines 3D optoacoustic with pulse-echo ultrasound imaging and allows for achieving deep, high-quality optoacoustic imaging.

Simultaneous optoacoustic and ultrasound imaging functionality is crucial for successful introduction of the new diagnostic imaging approach into the clinics. The novel transducer is capable of producing 3D optoacoustic images in parallel with a 2D ultrasound image while having a broader illumination pattern, and to integrate this novel transducer into the available clinical apparatus.

Preferably, ultrasound (US) imaging functionality is integrated into a 3D optoacoustic imaging probe, which imposes a number of technical challenges.

In contrast to pulse-echo ultrasound, efficient image acquisition in 3D optoacoustic imaging is achieved by means of specifically-designed spherical matrix arrays (first detection unit 23), with broad tomographic coverage around the imaged object. To increase sensitivity in detecting relatively weak optoacoustic responses, the individual elements are designed to have a large size; this makes them unsuitable for pulse-echo ultrasonography, which needs small element pitch for high resolution and artefact-free image rendering.

This is addressed by providing a multi-segment array transducer geometry combining spherical and linear array segments. This enables optimal imaging performance in both MSOT and US modes.

Parameter selection like size, pitch and distribution of the detector elements are preferably driven by simulations of different layouts.

Preferably, the development of the ultrasound detector arrays (combined US imaging with linear and spherical array segments) addresses the requirements of the specific application and especially the miniaturization challenge (hundreds of elements in a few centimeters diameter) and the integration challenge of the imaging array confined to the spherical geometry of the active surface.

The general configuration of the arrays may include, but is not limited to, array geometries (aperture, curvature radius, number of elements, element size and pitch), their frequency and the distribution of the elements.

The optoacoustic imaging part is optimized for sensitive detection with high signal to noise ratio (SNR) over broad frequency range while maximizing the available angular tomographic coverage. The array segment dedicated to pulse-echo US imaging is designed to enable high resolution imaging while minimizing sidelobe artefacts and maximizing contrast in the images.

Preferably, the geometry and/or mechanical/electrical properties of active piezoelectric materials (piezocomposites) and passive materials (backing, acoustic matching layers) and electrical components (transformer, capacitance or inductance) that could be used are taken into account. The properties of the active and passive materials are optimized in order to obtain the best performances (bandwidth, SNR, angular acceptance in receive mode).

Preferably, the US imaging array segment (second detection unit 24) is tightly integrated with the segment (first detection unit 23) dedicated to optoacoustic imaging. Dead zones between the two array segments are preferably minimized, because blind zones could impact the overall image quality. The active aperture and overall size of the transducer array is preferably minimized to allow for a compact (miniaturized) handheld design.

Preferably, piezocomposite materials are used to manufacture the prototypes.

Following configurations are particularly preferred:
A linear array segment (second detection unit 24) placed in the center of the spherical matrix array (first detection unit 24) consisting of the same piezocomposite material, the two parts being centered around the same frequency thus resulting in the most compact structure.
Alternatively, the linear array segment (second detection unit 24) can be constructed separately and integrated later on into a dedicated window inside the spherical matrix array (first detection unit 23). In this case, the linear array segment can have a higher frequency to enable both high resolution US imaging and deeper penetration in the MSOT mode with a lower detection frequency range. However, the gap (blind zone) between the two array segments would be larger in that case.
Outsourcing of the linear array segment (second detection unit 24) is another option, in which case a standard commercial product can be adapted for pulse-echo US imaging. This allows for further optimizing the 3D OA imaging performance of the transducer array (first detection unit 23).

The exact geometry of the spherical array segment (first detection unit 23) can be preferably determined based on simulation studies, however its aperture should preferably not exceed 4 cm. This may be addressed preferably by:
integration of a plurality, e.g. 512 or more, individual detection elements onto a relatively small and highly curved surface,
connection with the digitization electronics, including shielding for minimizing noise.

In particular, the arrays are preferably crafted onto a highly curved (<5 cm radius) surface, while on the other hand cracks are prevented, the final array geometry can be controlled and a consistent performance across the elements is delivered.

Dedicated illumination approaches are also preferred: several openings 22a in the active array surface of the first detection unit 23 are provided for inserting fiber bundles of the guiding element 22 (see FIG. 1a). The outer materials (front face, housing) preferably prevent light absorption and parasitic emission that would lead to image artefacts.

Preferably, high coupling coefficient piezoelectric materials (e.g. single crystals) are used for improving the effective bandwidth of the linear array segment. Preferably, advanced US field simulations may be employed in order to assess relevance of the design parameters to the intended clinical imaging application. Specific processes will be developed in order to allow the use of these materials for realizing the prototypes.

Preferably, the electrical impedance of the arrays is chosen to match to the electrical environment.

For the MSOT imaging part, the electro-acoustical behavior in receive mode may be investigated as follows:
the receive transfer function (ratio between the generated voltage and the received pressure) will be measured and the bandwidth will be deduced from its frequency spectrum
the acceptance angle will be measured
the Minimum Detectable Pressure (MDP) will be estimated in frequency and space from noise and receive transfer function measurements.

The beamforming capabilities of the arrays may be assessed with driving electronics in the lab before integration into the final system.

The desired hand-held probe miniaturization and deeper tissue penetration impose challenging requirements on both the ultrasound array design and the accompanying laser illumination solution. In order to achieve an effective 3 cm penetration depth in the MSOT imaging mode, per-pulse laser energies up to 100 mJ are preferably used at the OPO laser output, with the transducer aperture optimized for detection from deep tissue regions. To meet the laser safety standards for human skin exposure in the near-infrared range, the laser energy may be distributed over an area of >3 cm² on the patient's skin surface, either through multiple fiber bundles or by means of fiber illumination interleaved with the piezo elements. Further, numerical simulations of the entire light propagation path are preferred, with the overall goal of optimizing light delivery into the target imaged areas deep within scattering and absorbing living tissues. Moreover, an optimal casing 25a for the new imaging probe 25 may be preferred, the casing preferably incorporating the new array transducer comprising the first and second detection unit 23, 24 and multi-arm and/or transducer-interleaved light delivery system. The casing design preferably includes an acoustically and optically transparent coupling medium, thus mitigating signal losses and aberrations. Size and weight may be optimized for handheld imaging while also meeting the specific requirements of paediatric, small-contact-area imaging.

By means of the preferred detection unit simultaneous real-time acquisition and rendering of high quality volumetric MSOT and pulse-echo US images is achieved. The envisaged hybrid-mode handheld ultrasound probe 25 and accompanying electronics preferably requires recording of real-time data from a large number of elements. Such recordings may result in challenging requirements in terms of data acquisition/transfer rates, signal processing and memory storage, especially in case of lengthy imaging sessions.

These challenges are preferably met by a new class of sparse acquisition and compressed sensing (CS) algorithms that dramatically reduce the amount of data required for simultaneous real-time imaging and the corresponding data processing and storage requirements. These image reconstruction approaches are facilitated by implementation on CS-enabled random channel acquisition high-speed electronics. In developing the algorithms, the underlying high signal redundancies and sparsity in the spatial, temporal and spectral domains may be beneficial, such that the rendering of high frame-rate (>25 Hz) volumetric MSOT and pulse-echo US images using under-sampled data without compromising the resulting image quality and signal quantification abilities is enabled.

The preferred detection unit 25 is particularly suitable for determining geometry, acoustic and/or optical properties of muscle tissue and/or muscle regions, in particular for diagnosing DMD.

Preferably, the device, system and/or methods making use of the preferred detection unit 25 allows for delivering enhanced diagnostic accuracy based on the detection of biomarkers such as collagen, haemoglobin, myoglobin and lipids.

In addition, specific physical challenges when diagnosing very young patients are preferably addressed by:

(a) using an OPO (optical parametric oscillator) laser with significant smaller footprint to increase mobility of the system;

(b) reduction of laser safety requirements (to new laser class 1c), so that patients (especially infants) can be treated without the need to wear goggles;

(c) calibration of the new laser for repeated measurements over time, optimization of illumination, increasing detection depth and sensitivity;

(d) enhancing the transducer design to combine 3D optoacoustic imaging with pulse-echo ultrasound in a single probe 25;

(e) making the transducer physically smaller, so that contact with smaller limbs and bodies can be assured;

(f) enhancing the system data processing features (by new reconstruction algorithms for new detectors, real-time 3D image processing, image improvement through artefact removal) and

(g) improving post-processing and analysis by using artificial intelligence for automated image improvement, segmentation and selection of the region of interest (ROI) inside the image area and development of batch analysis algorithms for big data sets.

(h) demonstration and verification of system performance of the new prototype system in small cohorts of different muscular diseases by comparing to the existing pilot study and MRI data, and small-scale longitudinal clinical treatment feasibility studies.

In particular, the invention allows for monitoring patient response to therapeutic interventions and thus informing better clinical decision making in real world conditions.

### Collagen as Non-Invasive In Vivo Molecular Imaging Biomarker in Duchenne Muscular Dystrophy

Further preferred aspects of the invention, in particular with respect to using collagen as non-invasive in vivo molecular imaging biomarker in assessing and/or diagnosing and/or treating and/or monitoring Duchenne muscular dystrophy (DMD), are disclosed below.

DMD is the most common X-linked lethal muscular disease causing muscular fatty and fibrotic transformation with progressive loss of physical function. Until now, treatment response assessments are solely based on individual physical examinations.

According to a preferred application of the invention, it is possible to quantitate muscle collagen content using multispectral optoacoustic tomography (MSOT) for disease assessment in Duchenne muscular dystrophy. In an in-pediatric cross-sectional trial that included patients with DMD and matched healthy volunteers, mean collagen content in the muscles was found to be statistically significantly different between the groups (15.29±2.36 a.u. vs 25.05±2.66 a.u.). Furthermore, the collagen content correlated significantly with the clinical function as based on the 6-minute walk test (6-MWT). Thus, MSOT-derived collagen is a potential age-independent imaging biomarker for disease progress monitoring in DMD and a showcase for future applications.

In particular, it has been determined that i) extended near-infrared imaging with multispectral optoacoustic tomography (MSOT) enables detection of collagens in vivo, and ii) MSOT derived collagen can be used as a non-invasive biomarker for disease progression monitoring in Duchenne muscular dystrophy (DMD).

A translational feasibility study comprised i) a blinded case-control study of one to three days old piglets of a female carrier pig (DMD^{+/-}), and ii) a first-in-pediatric monocentric, open-label, parallelized clinical trial.

Participants of the study were the following: i) Seventeen piglets of three different litters (N=10 wild type (WT) and N=7 DMD piglets) ii) N=10 DMD patients, as ambulatory, male, and three to ten years of age and N=10 gender and age matched healthy volunteers (HV); HV with any pre-existing muscular disorder were excluded.

All participants underwent one-stage, non-invasive, in vivo 2D and 3D (children only) multispectral optoacoustic tomography (MSOT) of defined muscle regions.

The primary comparison was the muscle collagen content (mean and maximum) between the WT/HV and the DMD groups.

As a result, 2D MSOT is able to detect fibrotic muscular degeneration in DMD piglets and patients by means of increased collagen_MEAN/collagen_MAX signals compared to controls. Further, 3D MSOT demonstrates significantly increased collagen_MEAN/collagen_MAX, and decreased HbR and HbO2 signals in DMD patients compared to HV. The degree of collagen_MEAN/collagen_MAX corresponds with experimental histopathology and with human physical performance (e.g. 6-minutewalk-test vs. collagen_2 D-MEAN) showing independence from age (e.g. age vs. collagen_2 D-MEAN).

Preferably, in the study the images were obtained with a hybrid ultrasound MSOT system comprising a 25 Hz pulsed Nd:YAG laser and two detector units for optoacoustic and ultrasound wave detection. Multispectral optoacoustic tomography signals were acquired at 680, 700, 730, 760, 800, 850, 920, 1000, 1030, 1064, and 1100 nm.

Preferably, a 2D concave handheld detector (4 MHz center frequency, 256 transducer elements) with a field of view of 30 mm and spatial resolution of <150 μm provides cross sectional images and is combined with a reflective ultrasound computed tomography (RUCT) unit, to anatomically guide optoacoustic imaging during the examination. Preferably, a 3D hemispherical handheld detector (8 MHz center frequency, 256 transducer elements) with a field of view of 15 mm and a spatial resolution of 100 μm provides isotropic volumetric optoacoustic images. Transparent ultrasound gel (AQUASONIC Clear®, Parker Laboratories Inc., Fairfield, N.J., USA) was used for coupling between detector and skin. A polygonal region of interest (ROI) was placed just beneath the muscle fascia according to the MSOT-signal.

MSOT data were reconstructed with direct backprojection and spatial fluence correction was applied for images acquired with the 2D detector (μₐ=0.022 and μₛ=10 cm⁻¹). Using spectral unmixing, MSOT values for HbR, HbO2 and collagen were obtained. Collagen unmixing was based on acquired wavelengths of the entire spectral range, whereas HbR, HbO2 signal was calculated from a subrange (730 nm and 850 nm) which is more accurate in unmixing due to increase of water absorptivity at higher wavelengths. Single wavelength of 920 nm was used to depict lipid signals.

FIG. 7 shows a diagram illustrating in vivo 2D MSOT imaging of newborn piglets.

FIG. 7A shows a scheme of the handheld 2D concave MSOT detector probe (4 MHz center frequency), wherein i) ExNIR laser light is emitted to muscle tissue, ii) thermoelastic expansion of absorbers (e.g. collagen) generates ultrasound waves, and iii) ultrasound waves are detected by the detector probe.

FIG. 7B shows exemplary images of a healthy (WT) (upper row) and a DMD piglet (bottom row). Regions of interest (ROIs, boxes) are determined in the reflection ultrasound computed tomography images (RUCT, used to anatomically guide the investigator). Qualitative differences of spectrally unmixed optoacoustic collagen signals between WT and DMD piglets are shown in the respective MSOT image. The merged MSOT/RUCT image visualizes the collagen distribution within the muscle and the ROI, respectively. Scale bar indicates 5 mm.

FIG. 7C shows Hematoxylin & Eosin (H&E, 10-fold magnification), Masson's Trichrome (TriC, 10- and 40-fold magnification insert), and dystrophin (Dys1, 40-fold magnification) immunohistochemistry stainings from imaged piglet musculature (WT upper row and DMD piglet bottom row). H&E shows disrupted muscular structure, TriC increased collagen content, and Dys1 dystrophin expression pattern. Black boxes represent sites for higher magnification. Scale bars indicate 100 μm.

FIG. 7D shows pooled mean and maximal collagen signals of both groups demonstrating statistically significant signal differences between WT and DMD piglets.

N=17 piglets underwent standardized MSOT imaging: transversal scans of the shoulder (M. triceps brachii) and the leg (M. biceps femoris) muscles. A schematic description of the MSOT imaging principle is presented in FIG. 7A.

A 2D imaging approach was developed in a translational porcine model of DMD. In total N=112 scans (N=64 in WT and N=48 in DMD) were acquired from N=17 piglets (WT, N=10 and DMD, N=7). Using ultrasound guidance, a region of interest (ROI) was drawn within the examined muscle to quantify the MSOT parameters (FIG. 7B). Ex vivo histopathology revealed muscular dystrophy and a qualitative increase in collagen formation in diseased animals (FIG. 7C). Using spectral unmixing, the mean and the maximum collagen signals within the ROI were calculated. When compared to WT, the pooled collagen signal from all muscle regions of DMD piglets was significantly increased (collagen_MEAN 14.22±1.96 vs. 22.68±3.48; collagen_MAX 27.69±1.68 vs. 40.84±4.88), see FIG. 7D. There was no difference in signal levels of deoxygenated, oxygenated and total hemoglobin. An exploratory receiver operator characteristic (ROC) analysis demonstrated excellent ability of MSOT-derived collagen signals to distinguish healthy from diseased piglets.

FIG. 8 shows a diagram illustrating in vivo 2D MSOT imaging and collagen quantification in DMD patients and healthy volunteers (HV).

FIG. 8A shows an exemplary photo of real-time imaging of a 3-year old HV with the 2D MSOT detector probe.

FIG. 8B shows exemplary RUCT and MSOT images of transversal scans from four anatomical regions of a 7-year-old healthy volunteer (HV, left panels) compared to a 5-year-old DMD patient (DMD, right panels). RUCT images were obtained for anatomically guidance during examination. MSOT/RUCT merged images show MSOT signals for hemoglobin and collagen, preferably as color-coded maps, overlayed on the gray-scaled RUCT image. A qualitative difference of collagen signal intensity was found in every muscle region between both groups. Boxes in the RUCT images indicate regions of interest used for signal quantification.

FIG. 8C shows normalized optoacoustic spectra of collagen of a DMD patient (c), a HV (b), a ratio (a) between DMD patient and HV and the literature (d). The collagen spectra found in DMD patients were consistent with the spectra found in the literature (collagen peak indicated at 1000 nm).

FIG. 8D shows the pooled mean and maximal collagen signal (a.u.) indicating significant differences when comparing HV and DMD patients.

FIG. 9 shows a diagram illustrating in vivo 3D MSOT imaging in DMD patients and healthy volunteers.

FIG. 9A shows a scheme of the handheld 3D hemispherical MSOT detector probe (8 MHz center frequency).

FIG. 9B shows 3D images of the same two boys from FIG. 8. Top row: 7-year-old healthy volunteer (HV). Bottom row: 5-year-old DMD patient (DMD). Maximum projection images of the gastrocnemius muscle in two axes (XZ and YZ) and a 3D volumetric (volume) area are depicted with color-coded maps of HbT, collagen_MEAN and lipid.

FIG. 9C shows an example for quantification of 3D MSOT parameters (HbR=deoxygenated hemoglobin, HbO2=oxygenated hemoglobin, HbT=total hemoglobin, C_MEAN=collagen_MEAN, C_MAX=collagen_MAX). Black dots represent HV and grey dots represent DMD patients.

In total N=320 scans (transversal and longitudinal scans of eight muscles per participant) were obtained. The average scan time for 2D and 3D images was 6.3±0.8 min in DMD patients and 6.7±0.6 min in HV.

An example of real-time in vivo 2D MSOT imaging is presented in FIG. 8A. Further, exemplary transversal images of four anatomical regions (forearm flexors, biceps-, quadriceps-, and gastrocnemius muscle) of a single HV and DMD patient are presented in FIG. 8B. The MSOT-derived collagen spectrum extracted from the patient's data closely resembled the expected spectrum found in the literature (FIG. 8C). In accordance to a preclinical model, each muscle was analyzed for its mean and maximum collagen content. All muscle regions showed statistically significant differences between both groups for every MSOT collagen parameter. Again, after pooling signal levels of all anatomical regions significant differences were found in the mean (collagen_MEAN 15.29±2.36 a.u. vs 25.05±2.66 a.u.) and the maximum collagen content (collagen_MAX 27.62±3.06 a.u. vs 40.17±3.18 a.u. (see FIG. 8D). There was no difference found in lipid (68.47±4.92 a.u. vs 71.95±6.60 a.u.), deoxygenated hemoglobin (HbR 30.55±6.49 a.u. vs 28.86±5.28 a.u.), oxygenated hemoglobin (HbO2 60.05±5.55 a.u. vs 60.86±3.61 a.u.) and total hemoglobin (HbT 89.55±11.06 a.u. vs. 89.72±7.85 a.u.) signals between both groups.

In total N=320 ultrasound images were evaluated and all HV showed hypoechogenic, inhomogeneous and medium to coarse granular muscles (Heckmatt scale: 1). In DMD patients it was found N=68 (42%) hyper-echogenic, N=2 (1%) homogeneous, N=18 (11%) focal, N=24 (15%) finegranular muscle alteration and 52 (33%) muscles were scored 2 or higher on Heckmatt scale. A weak correlation between collagen (2D collagen_MEAN) and Heckmatt scale derived from ultrasound images was found (Pearson r=0.34).

For 3D MSOT collagen imaging, a total of N=160 3D scans were acquired. While the subcutaneous fat signal is similar, collagen signals differ clearly between the groups. The detection of the subcutaneous tissue is enabled by the design of the 3D detector probe (FIGS. 9A and 9B). Similar to the above-mentioned 2D analysis approach, 3D data sets demonstrated a significant difference of the mean and maximum collagen content in all anatomical regions. Pooled mean and maximum collagen signal for each participant were analyzed and showed significant differences (collagen_MEAN 5.42±0.81 a.u. vs 11.39±1.02 a.u. and collagen_MAX 12.95±1.18 a.u. vs 23.84±3.13 a.u.) (see FIG. 9C).

Notably, significant differences were found in the optoacoustic signals for HbR and HbO2 acquired with the 3D detector. In DMD muscles, HbR and HbO2 were significantly decreased (HbR 9.13±2.38 a.u. vs 5.86±1.90 a.u.; HbO2 15.70±3.63 a.u. vs 7.47±1.18 a.u.) (see FIG. 9C). The mean collagen content correlated negatively with deoxygenated hemoglobin and oxygenated hemoglobin contents of the muscle.

Further, significant negative correlations were found between every MSOT collagen parameter and the 6-MWT (collagen_2 D-MEAN −0.75; collagen2D-MAX −0.74; collagen_3 D-MEAN −0.73; collagen_3 D-MAX −0.73). The other timed function tests (Rise from supine, 10 m walk/run, 4 stairs climb, 8 stairs climb and sit to stand) and manual muscle testing (MRC) showed a consistent correlation with collagen signals, except single rise from chair and MRC of the lower distal extremity correlated with collagen_MAX. While timed function tests and manual muscle testing significantly correlated with MSOT collagen signals, a correlation towards individual age was not observed.

FIG. 10 shows a table giving an overview of piglet 2D-MSOT parameters, i.e. pooled (all muscle regions of each piglet) MSOT signals. Multispectral unmixing for MSOT collagen parameters (collagen_MEAN and collagen_MAX) was derived from all acquired wavelengths (680, 700, 730, 760, 800, 850, 920, 1000, 1030, 1064, 1100 nm). Multispectral unmixing for MSOT hemoglobin parameters (HbR, HbO2, HbTotal) was derived from a subrange (730 nm and 850 nm), which is more accurate in unmixing due to increase of water absorptivity at higher wavelengths. Whereas collagen_MEAN/collagen_MAX showed statistically significant differences between WT and DMD-piglets, no difference of the hemoglobin_R/O2/Total content between cohorts was found.

FIG. 11 shows a table giving an overview of mean and maximum 2D-MSOT collagen signal sorted by scanning region. Multispectral unmixing for MSOT collagen parameters (collagen_MEAN and collagen_MAX) was derived from all acquired wavelengths (680, 700, 730, 760, 800, 850, 920, 1000, 1030, 1064, 1100 nm). All regions showed statistically significant differences between HV and DMD-patients.

FIG. 12 shows a table giving an overview of human 2D-MSOT parameters, i.e. pooled (all muscle regions of each participant) MSOT signals. Multispectral unmixing for MSOT collagen parameters (collagen_MEAN and collagen_MAX) was derived from all acquired wavelengths (680, 700, 730, 760, 800, 850, 920, 1000, 1030, 1064, 1100 nm). Multispectral unmixing for MSOT hemoglobin parameters (HbR, HbO2, HbTotal) was derived from a subrange (730 nm and 850 nm), which is more accurate in unmixing due to increase of water absorptivity at higher wavelengths. Single wavelength of 920 nm was used to depict lipid signals. Whereas collagen_MEAN/collagen_MAX showed statistically significant differences between HV and DMD-patients, no difference of the hemoglobinR/O2/Total and lipid content between cohorts was found.

FIG. 13 shows a table giving overview of human 3D-MSOT parameters, i.e. pooled (all muscle regions of each participant) MSOT signals. Multispectral unmixing for MSOT collagen parameters (collagen_MEAN and collagen_MAX) was derived from all acquired wavelengths (680, 700, 730, 760, 800, 850, 920, 1000, 1030, 1064, 1100 nm). Multispectral unmixing for MSOT hemoglobin parameters (HbR, HbO2, HbTotal) was derived from a subrange (730 nm and 850 nm), which is more accurate in unmixing due to increase of water absorptivity at higher wavelengths. All MSOT parameters showed statistically significant differences between HV and DMD-patients.

In summary, the disclosed translational approach suggests a potential role for MSOT as a novel in vivo contrast-agent free and non-invasive imaging modality for the quantitative detection of collagen as a biomarker in DMD.

The utilization of wavelengths in the extended near infrared range (exNIR) has so far not been applied in a clinical setting—especially not in pediatrics. The findings disclosed herein suggest quantitative assessment of collagen content in muscles with MSOT for monitoring of degeneration involving fibrotic processes in vivo, as shown in this study in a large animal model. The extracted collagen spectra were in agreement with previously reported optoacoustic spectra; visualization and quantification of collagen was feasible in all anatomical regions and showed statistically significant differences between diseased and healthy animals as well as in the human cohorts. Accelerated disease progression in DMD piglets leading to early structural changes might allow comparison of animal and human findings in this study.

Currently several new therapeutic treatments for DMD are under investigation, but until now, there is an unmet need for established objective monitoring techniques and age-independent biomarkers in clinical practice. So far, the 6-MWT is the most commonly used primary endpoint for disease assessment in DMD but essentially requires active patient compliance. The complexity to accurately execute these tests and dependence on cooperative behavior limit the significance of muscular function tests to more adolescent patients. Most recent trials were restricted to DMD patients aged over 5-7 years, which prohibits conclusions on early therapeutic interventions. In comparison to established imaging modalities like ultrasound imaging and MRI (magnetic resonance imaging) MSOT is a quantitative, non-invasive, bedside imaging modality. MRI, which showed a potential capability of treatment monitoring, has a long image-acquisition time and usually causes discomfort, requires immobilization and, in early childhood, sedation. In the disclosed study it is demonstrated that MSOT can be performed in subjects down to 3 years of age, while minimal scan times suggest that it could even be performed at any postnatal age.

The slightly divergent imaging outcomes for the 2D and 3D detector might reflect different technical designs of the detectors used in the study. The differences of deoxygenated and oxygenated hemoglobin content of the muscle between HV and DMD patients and the negative correlation of hemoglobin and collagens is in line with the pathophysiological mechanism of muscular degeneration underlying DMD. Notably, fatty transformation could not be detected using MSOT, most likely, due to the high absorption of the subcutaneous fat tissue.

As it was the first-in pediatric use of multispectral optoacoustic imaging, the disclosed study is limited by its feasibility design and therefore, by its number of participants. Nevertheless, N=320 2D scans and N=160 3D scans were recorded in this exploratory study, which underline the ability of MSOT as a highly specific and sensitive diagnostic tool for DMD.

The study demonstrates an age-independent, highly statistically significant negative correlation between MSOT collagen parameters and the clinical muscle function. Novel causal therapeutic approaches leading to ultrastructural restoration of damaged muscles could therefore be directly visualized using MSOT in future studies. The disclosed approach reaching from experimental tissues to first in-patient application supports MSOT-derived collagen detection and quantification as a potential age-independent imaging biomarker for disease progress monitoring in DMD.

In the following, preferred embodiments, elements, features, characteristics, advantages and/or alternatives of an optoacoustic system, an optoacoustic device, a method and a computer program according to at least one of the aspects a) to z) disclosed above are described.

Optoacoustic tomography systems attain unprecedented volumetric imaging speeds, thus enabling insights into rapid biological dynamics and marking a milestone in the clinical translation of this modality. Fast imaging performance often comes at the cost of limited field-of-view, which may hinder potential applications looking at larger tissue volumes. The imaged field-of-view can potentially be expanded via scanning and using additional hardware to track the position of the imaging probe. However, this approach is challenging for high-resolution volumetric scans performed in a freehand mode along arbitrary trajectories.

Therefore, an accurate framework for spatial compounding of time-lapse optoacoustic data is preferably used, preferably in combination with aspects of the device and method for analyzing optoacoustic data, optoacoustic system for generating and analyzing optoacoustic data disclosed herein.

The method preferably exploits the frequency-domain properties of structures, preferably vascular networks, in optoacoustic images and estimates the relative motion and orientation of the imaging probe. This allows for rapidly combining sequential volumetric frames into large area scans without additional tracking hardware. The approach is universally applicable for compounding 2D or volumetric (3D) data acquired with calibrated scanning systems but also in a freehand mode with up to six degrees of freedom. Robust performance is demonstrated for whole-body mouse imaging with spiral volumetric optoacoustic tomography and for freehand visualization of vascular networks in humans using volumetric imaging probes. The newly introduced capability for angiographic observations at multiple spatial and temporal scales is expected to greatly facilitate the use of optoacoustic imaging technology in pre-clinical research and clinical diagnostics, in particular in combination with aspects of the present disclosure. The technique can equally benefit other biomedical imaging modalities, such as scanning fluorescence microscopy, optical coherence tomography or ultrasonography, thus optimizing their trade-offs between fast imaging performance and field-of-view.

The method, system, device and according computer program is preferably based on the approach of analyzing optoacoustic data by: s1) receiving optoacoustic data from a probe, in particular a handheld probe, wherein the optoacoustic data relate to acoustic waves which were generated in a tissue, in particular comprising muscle tissue, in response to an irradiation of the tissue with time-varying, in particular pulsed, electromagnetic radiation at two or more different irradiation wavelengths and detected by a concave, in particular spherically shaped, matrix array of transducer elements of the probe while the probe is being moved, in particular translated and/or rotated, to a plurality of different probe positions, in particular including different locations and/or orientations of the probe, relative to the tissue, s2) reconstructing, in particular 3D, optoacoustic images (also referred to herein as “single-wavelength optoacoustic images” or “single-wavelength MSOT images”) based on the optoacoustic data relating to the acoustic waves which were detected at the different irradiation wavelengths and probe positions so as to obtain, for each of the different irradiation wavelengths, a sequence of optoacoustic images (iₙ, n=1 . . . N), which were obtained for the different probe positions, s3) generating, for each of the different irradiation wavelengths, a compounded, in particular 3D, optoacoustic image (iₛ) from the sequence of optoacoustic images (iₙ, n=1 . . . N) by i) determining a location (Tₙ, n=2 . . . N) and/or rotation (θₙ, n=2 . . . N) of at least one optoacoustic image (iₙ) of the sequence relative to an initial optoacoustic image (i₁) of the sequence, and ii) combining the at least one optoacoustic image (iₙ) of the sequence with the initial optoacoustic image (i₁) of the sequence so as to obtain the compounded optoacoustic image (iₛ) by considering the determined location (Tₙ) and/or rotation (θₙ) of the at least one optoacoustic image (iₙ) of the sequence relative to the initial optoacoustic image (i₁) of the sequence, and s4) determining at least one, in particular 3D, concentration image (also referred to as “MSP image”) relating to a, in particular three-dimensional, spatial distribution of a concentration (also referred to as “first value”) of a substance, in particular a component and/or biomarker such as collagen, in the tissue based on the compounded optoacoustic images generated for the different irradiation wavelengths.

According to this approach, motion of a freehand probe between consecutive frames (images) can be estimated from the imaging data in order to obtain a compounded optoacoustic image iₛ of a large volume of the tissue under investigation.

In the following, the framework for spatial compounding of time-lapse OA data acquired using large-area volumetric scans is described in more detail. The method takes advantage of the frequency-domain properties of structural, in particular vascular, networks in OA images, which allows for optimally combining sequential volumetric frames into large area scans without using additional tracking hardware.

Preferably, vascular patterns can be usually identified in volumetric OA images acquired by the spherical-array-based (3D) probe(s) disclosed herein.

It is noted, however, that the framework for spatial compounding of OA images disclosed herein does not necessarily require OA images of a tissue containing vascular structures. Rather, any structure within the imaged volume or tissue (i.e. skin or skin layers, fatty tissue, connective tissue, muscle etc.) may be analyzed regarding frequency-domain properties as described in in more detail below.

FIG. 14 shows a schematic diagram illustrating a Fourier-based 3D motion estimation. (a) Lay-out of the 3D opto-acoustic tomography probe used in the experimental study, indicating the 6 degrees of freedom corresponding to an arbitrary freehand motion. (b) 2D rotation estimation is based on the maximum intensity projections (MIPs) of the volumetric image frames (step 1), which are reduced to their brightest pixels (step 2). After applying Fourier transform (step 3) and resolution enhancement (step 4), the spectra are correlated for different rotation angles, yielding a RCF for x-, y- and z-axis. Rotation parameters are extracted after least squares approximation (LSA) of the RCF (dashed line). (c) Translation estimation based on two consecutive frames i1 in blue and i2 in red, respectively (step 1). The frames are reduced to their relevant structures, represented by the brightest voxels (step 2) before the PoC is computed (step 3, equations. 8 and 9). The translation parameters are finally extracted after Gaussian noise suppression (step 4).

OA images can be acquired with a spherical matrix ultrasound array schematically depicted in FIG. 14a. Briefly, an 8 cm diameter spherical array consists of 256 or more densely distributed piezocomposite elements having 4 MHz central frequency and −100% (half width at half maximum) detection bandwidth, resulting in an effective point spread function (spatial resolution) of 200 μm around the center of the spherical array geometry. Optical excitation was provided with an optical parametric oscillator (OPO) laser guided with a fiber bundle through a central cavity of the array. The laser further provides fast wavelength tunability between 680 and 950 nm on a per pulse basis and an additional 1064 nm beam output. A custom-made data acquisition system, triggered with the Q-switch output of the laser, was used to digitize the detected individual pressure waveforms at 40 megasamples/s and simultaneously transfer them via a 1 Gbps Ethernet connection to PC for further processing and storage. The acquired signals were deconvolved with the electric impulse response of the piezoelectric detection elements, band-pass filtered with cut-off frequencies of 0.1 and 6.0 MHz and subsequently processed with a back-projection reconstruction procedure, as described in X. L. Dean-Ben, A. Ozbek, and D. Razansky, “Volumetric RealTime Tracking of Peripheral Human Vasculature With GPU-Accelerated Three-Dimensional Optoacoustic Tomography,” IEEE Trans. Med. Imaging, vol. 32, no. 11, pp. 2050-2055, November 2013. A volume of 12 mm×12 mm×12 mm containing 120×120×120 voxels was reconstructed for each acquired frame.

The relative position and orientation of consecutive 3D images acquired with a real-time OA scanner following an arbitrary scanning trajectory can in a general form be expressed as superposition of translations and rotations. One may represent the translation by a vector t=[tₓ,t_{y},t_{z}]^{T} describing the displacements in x-, y- and z-direction, whereas the rotation may be represented by a vector θ=[θₓ,θ_{y},θ_{z}]^{T} describing the rotation angles around the corresponding axes (FIG. 14a). This yields a total of six degrees of freedom for any arbitrary motion.

Rotation among subsequent acquisitions can be estimated by cross-correlating rotated Fourier spectra and selecting the rotation angle that attains the best fit, which is preferably based on the PROPELLER method used in MRI and generalized for 3D images and rotations around more than one axis. Let i₁(x,y,z) and i₂(x,y,z) be sequentially acquired images having a sufficient degree of spatial overlap yet acquired at different position and orientation. The 3D-Fourier transform I₂(fₓ,f_{y},f_{z}) of i₂(x,y,z) is rotated around different angle combinations θ=[θₓ,θ_{y},θ_{z}]^{T} (represented by I₂^{θ}(fₓ,f_{y},f_{z})) and cross-correlated with the Fourier transform I₁(fₓ,f_{y},f_{z}) of i₁(x,y,z). As similarity metric, the normalized cross-correlation (NCC) is calculated according to Ashish A. Tamhane and Konstantinos Arfanakis, “Motion Correction in PROPELLER and Turboprop-MRI,” Magn Reson Med, vol. 62, no. 1, pp. 174-182, 2009, and referred to as the rotation correlation function (RCF):

$$RCF ( {θ}_{x} , {θ}_{y} , {θ}_{z} ) = \frac{1}{\sqrt{{P}_{1} {P}_{2}}} \int \int \int \vert {I}_{1} ( {f}_{x} , {f}_{y} , {f}_{z} ) \cdot {I}_{2}^{θ} ( {f}_{x} , {f}_{y} , {f}_{z} ) \vert d ( {f}_{x} , {f}_{y} , {f}_{z} ) , \qquad ( 1 )$$

where P1 and P2 are the image powers (average quadratic image intensity) of frames i₁(x,y,z) and i₂(x,y,z), respectively. Note that RCF(θₓ,θ_{y},θ_{z}) is not affected by phase differences associated with translational shifts. Any arbitrary rotation between frames is accurately estimated as

$$\hat{θ} = {[ {\hat{θ}}_{x} , {\hat{θ}}_{y} , {\hat{θ}}_{z} ]}^{T} = {argmax}_{{θ}_{x} , {θ}_{y} , {θ}_{z}} \{ RCF ( {θ}_{x} , {θ}_{y} , {θ}_{z} ) \} \qquad ( 2 )$$

As equations (1) and (2) represent a computationally expensive full 3D approach which may be challenging for high-resolution data, an approximated method is preferred, which uses maximum intensity projections (MIPs) of the 3D images as an input. According to Euler's rotation theorem, the rotation along an arbitrary direction is equivalent to three subsequent rotations along the corresponding

Cartesian coordinates. For small rotation angles (|θᵢ|<5°, i={x,y,z}), the coordinates (x′,y′,z′) of a point (x,y,z) after rotation can be approximately calculated using the simplified rotation matrix, i.e.

$$[ \begin{matrix}{x}^{′} \\ {y}^{′} \\ {z}^{′}\end{matrix} ] = [ \begin{matrix}1 & - {θ}_{z} & {θ}_{y} \\ {θ}_{z} & 1 & - {θ}_{x} \\ - {θ}_{y} & {θ}_{x} & 1\end{matrix} ] [ \begin{matrix}x \\ y \\ z\end{matrix} ] \qquad ( 3 )$$

One may subsequently estimate how the rotation along an arbitrary axis affects the MIP views of the object along the three Cartesian axes. Without loss of generality, the top view (along the z direction) of a 3D structure is considered first. According to (3), the new coordinates (x′,y′) of any point in the image can be estimated from the previous (x,y,z) coordinates of this point (before rotation) via

$$[ \begin{matrix}{x}^{′} \\ {y}^{′}\end{matrix} ] = [ \begin{matrix}1 & - {θ}_{z} \\ {θ}_{z} & 1\end{matrix} ] [ \begin{matrix}x \\ y\end{matrix} ] + [ \begin{matrix}{θ}_{y} \\ - {θ}_{x}\end{matrix} ] z \qquad ( 4 )$$

The first term in (4) represents a rotation around the z-axis. The second term may have different effects depending on the orientation of the particular structure. In the visible and near-infrared excitation spectra, endogenous OA contrast is mainly governed by hemoglobin with blood vessels appearing as the predominantly visible structures in the images. For vessels distributed normal to the z direction, the second term in (4) is constant, solely representing translation along x and y. For other vessels also expanding along z, this second term corresponds to non-rigid motion leading to shape distortions as manifested in the top views. As a result, vessels having dominant propagation directions in the x-y plane will be the main overlapping structures in the z-axis MIPs before and after rotation. Following the same reasoning, the correlation of the x- or y-axis MIPs before and after rotation will be mainly for the blood vessels expanding perpendicular to x and y, respectively. The rotation correlation function in (1) can be adapted for a faster MIP-based methodology via

$$R C {F}_{x} ( {θ}_{x} ) = \frac{1}{\sqrt{{P}_{1} {P}_{2}}} \int \int \int \vert {I}_{1 , MIP , x} ( {f}_{y} , {f}_{z} ) \cdot {I}_{2 , MIP , x}^{θ x} ( {f}_{y} , {f}_{z} ) \vert d ( {f}_{y} , {f}_{z} ) \\ R C {F}_{y} ( {θ}_{y} ) = \frac{1}{\sqrt{{P}_{1} {P}_{2}}} \int \int \int \vert {I}_{1 , MIP , y} ( {f}_{x} , {f}_{z} ) \cdot {I}_{2 , MIP , y}^{θ y} ( {f}_{x} , {f}_{z} ) \vert d ( {f}_{x} , {f}_{z} ) \\ RC {F}_{z} ( {θ}_{z} ) = \frac{1}{\sqrt{{P}_{1} {P}_{2}}} \int \int \int \vert {I}_{1 , MIP , z} ( {f}_{x} , {f}_{y} ) \cdot {I}_{2 , MIP , z}^{θ z} ( {f}_{x} , {f}_{y} ) \vert d ( {f}_{x} , {f}_{y} ) \qquad ( 5 )$$

where I_{1,MIP,i}(fⱼ,fₖ) and I^{θi}_{2,MIP,i}(fⱼ,fₖ) are the respective 2D Fourier transforms of the MIPs along the i-th direction before and after rotation. Note that the correlation functions in (5) generally depend on θ=[θₓ,θ_{y},θ_{z}]^{T}. However, as previously alluded, the correlation is primarily associated with a rotation around the Cartesian axis corresponding to the MIP view and it is further assumed that the correlation functions are solely dependent on this angle. The rotation angles along the x-, y- and z-directions are consequently estimated via

$${\hat{θ}}_{x} = {argmax}_{{θ}_{x}} \{ R C {F}_{x} ( {θ}_{x} ) \} \\ {\hat{θ}}_{y} = {argmax}_{{θ}_{y}} \{ RC {F}_{y} ( {θ}_{y} ) \} \\ {\hat{θ}}_{z} = {argmax}_{{θ}_{z}} \{ RC {F}_{z} ( {θ}_{z} ) \} \qquad ( 6 )$$

The two-dimensional Fourier-based estimation of the rotation angles is showcased in FIG. 14b including the image filtering steps introduced in the Image filtering section.

After the rotation between two consecutive frames has been estimated and compensated for, the translation estimation is performed by adapting a phase correlation method, preferably according to R. Szeliski, Image Alignment and Stitching: A Tutorial, vol. 2, no. 1, 2006, with frame i2(x,y,z) being merely a shifted version of frame i₁(x,y,z). By further taking into account the noise n(x,y,z) and interfering components d(x,y,z), which represent artifacts and transient structures either entering or leaving the effective FOV of the imaging system, the relationship between the two images is expressed as

i₂=i₁(x−tₓ,y−t_{y},z−t_{z})+n(x,y,z)+d(x,y,z) (7)

This relation is transformed into Fourier domain in order to compute the phase correlation function (PCF) defined as

$$P C F ( {f}_{x} , {f}_{y} , {f}_{z} ) = \frac{{I}_{1} \{ {f}_{x} , {f}_{y} , {f}_{z} ) \cdot {I}_{2}^{*} ( {f}_{x} , {f}_{y} , {f}_{z} )}{\vert {I}_{1} ( {f}_{x} , {f}_{y} , {f}_{z} ) \cdot {I}_{2}^{*} ( {f}_{x} , {f}_{y} , {f}_{z} ) \vert} \qquad ( 8 )$$

where the term I₂*(fₓ,f_{y},f_{z}) is the complex conjugate of I₂(fₓ,f_{y},f_{z}). The inverse Fourier transform of the PCF yields the phase only correlation (PoC), which corresponds to a Dirac's delta function at translation t, provided the images are not severely affected by noise or interfering transient structures. In present case, these terms reappear as disturbing components ñ and \tilde{d} in the PoC, i.e.

PoC(x,y,z)= [symbol] ⁻¹{PCF(fₓ,f_{y},f_{z})}=δ(x−tₓ,y−t_{y},z−t_{z})+ñ(x,y,z)+\tilde{d}(x,y,z) (9)

Assuming that the interfering components are negligible in comparison to the Dirac's delta function δ(x-t), the translation t can be estimated from the maximum of PoC, i.e.

$$\hat{t} = {[ {\hat{t}}_{x} , {\hat{t}}_{y} , {\hat{t}}_{z} ]}^{T} = {argmax}_{x , y , z} \{ PoC ( x , y , z ) \} \qquad ( 10 )$$

A schematic description of the method for translation estimation is depicted in FIG. 14c including image filtering steps.

OA images are known to be affected by noise, negative values associated to limited-view artifacts as well as reflection and reverberation artifacts, which propagate into the Fourier transform of the images. In order to increase the accuracy of both RCF and PoC computation, the 3D images were thresholded so that only the brightest voxels in every image are considered, i.e. 0.1% of voxels with the highest intensity representing the most dominant image features. The noise and undesired artifacts below the threshold levels are discarded (step 2 in FIGS. 14b-c). This measure has an additional benefit of eliminating distortions due to an inhomogeneous laser light distribution since the shift and rotation parameters are determined according to geometrical shapes, such as sharp edges along thresholded image features, rather than image intensities.

Since most of the spectral energy in the images is concentrated at low frequencies, only spectrally relevant components were selected for the rotation estimation. The spectral resolution in this region was furthermore enhanced by artificially increasing the number of samples in the Fourier domain via zero-padding of the MIPs (FIG. 14b, step 3 and 4). The RCF generally has a concave shape with a global maximum. However, in practice it is typically affected by noise, which may shift the actual position of the maximum. To attain a more accurate and robust estimation, a least squares approximation (LSA) of the RCF was performed by means of a higher order polynomial (FIG. 14b, step 5).

Preferably, a Gaussian low-pass filter is further applied to the PoC in order to mitigate noise associated with ñ and \tilde{d}. In case the PoC does not represent a Dirac's delta function but is distributed over multiple spatially neighboring positions, the low-pass filtering concentrates the distributed energy in its spatial focus, leading to a more accurate estimate based on the filtered PoC (FIG. 14c, step 4).

In total, N−1 rotations θₙ and translations tₙ between subsequent frames need to be estimated in order to render a compounded image iₛ from an OA dataset containing N sequentially acquired frames iₙ (n=1 . . . N). For this, the initial frame i, is assigned with coordinates T₁=[0,0,0]^{T} and orientation θ₁=[0,0,0]^{T}. Since translation and rotation can be superimposed, all the following frames iₙ (n=2 . . . N) are assigned with coordinates Tₙ=Σ_{i=2 . . . n} tᵢ and orientation θₙ=Σ_{i=2 . . . n} θᵢ. In essence, Tₙ represents the 3D trajectory of the transducer motion.

Note that the absolute position and orientation of every frame depends on all previous estimates represented by the relative rotation and shift between consecutive frames. Therefore, the estimation inaccuracies and error propagation are preferably further mitigated by an extra estimation step comparing every frame with the compounded image iₛ.

Once the location and orientation of frame iₙ is determined, it will be added to iₛ. For this, frame iₙ is first rotated to the original orientation of θ₁ and then combined with iₛ according to its position Tₙ. Here the image intensities have simply been added up, even though the redundant intensity values in overlapping frame areas can be combined in several other ways.

Preferably, the data/image processing and compounding methods can, e.g., be implemented in MATLAB (Mathworks Inc, Natick, Mass., USA) running on a 3.4 GHz Intel i7 3820 CPU with 64 GB of RAM.

FIG. 15 shows a schematic diagram illustrating spatial image compounding results for phantom scans. (a) The printed vessel-mimicking structure. (b) Maximum intensity projections (MIP) of a single 3D image frame taken for a single position of the detection array. (c) The corresponding MIP of the compounded volume. (d) Reference image of the microsphere phantom taken by a bright-field microscope. The corresponding MIPs of the single 3D frame and compounded image are shown in (e) and (f), respectively.

The Fourier-based motion estimation method described above was experimentally tested with three independent experiments. First, two phantoms were imaged in a handheld experiment in order to qualitatively validate the accuracy of the motion estimation method. The first phantom consisted of a vessel-mimicking structure with an approximate size of 30 mm×30 mm printed with black ink on a white paper and embedded in agar (FIG. 15a). In the second phantom, light absorbing polyethylene microspheres with ˜100 μm diameter (Cospheric BKPMS 90-106) were randomly distributed in an agar-based substrate (FIG. 15d). The phantoms were scanned by the spherical array probe in a freehand mode by following a random trajectory. The laser was tuned to operate at 720 nm wavelength and 10 Hz pulse repetition frequency. The probe was moved slowly with inter-frame displacements not exceeding several millimeters, thus ensuring sufficient overlap between the consecutive frames.

FIG. 16 illustrates a Fourier-based spatial compounding for a spiral volumetric optoacoustic tomography (SVOT) scan. (a) Schematic of the SVOT system showing scanning trajectory of the spherical matrix detection array. The inset shows a single reconstructed image frame covering an approximate volume of 1 cm³. (b) The known and estimated scanning positions of the center of the image frames are indicated by blue and red dots, respectively for four different slices (z-coordinates) of the scan. The exact location of the slices is labeled with blue dashed lines in panels f) and g). (c) MIP image (along the central mouse axis) of the compounded volume based on the estimated array positions and orientations. (d) The corresponding MIP rendered using the known array positions and orientations. (e) comparison of the rotation estimation accuracy in the SVOT scan for the 2D and 3D Fourier-based spatial compounding approaches. Standard deviation (STD) values in degrees are provided. (f) and (g) are 3D images of the compounded volume based on the estimated and known array positions and orientations, respectively. (h) and (i) are zoom-ins into the kidney area in (f) and (g).

In the second experiment, a spiral volumetric optoacoustic tomography (SVOT) scan of a female athymic nude-Fox1nu mouse (Harlan Laboratories LTD, Itingen, Switzerland) was performed in order to assess accuracy of the suggested motion estimation method. In this case, the orientation and position of every frame is known in advance, which provides a gold-standard reference for validating the algorithm's performance. For this purpose, the spherical matrix transducer array was translated and rotated around the mouse using calibrated stages in steps of 1.5 mm and 3°, respectively (FIG. 16a). The scan consisted of a total of 21 elevational positions and 61 angular positions. The 1064 nm output of the pulsed laser was used for OA signal excitation. During the experiment, the mouse remained in a stationary vertical position inside a water tank heated to 34° C.

Lastly, imaging of an arm and palm of a healthy volunteer was performed. For this, the spherical matrix transducer array was first translated across the imaged area using a mechanical stage covering a zigzag-type sampling pattern with 2 mm distance between neighboring grid points, thus acquiring a total of 21×21 volumetric image frames. A freehand scan was subsequently performed with the transducer slowly moved over the palm along an arbitrary trajectory with <2 mm inter-frame displacements. Total of 200 volumetric frames were recorded. For the human experiments, the wavelength was set to 800 nm and the laser was operated at pulse repetition frequency of 10 Hz. The laser fluence and average intensity at the skin surface were maintained below 15 mJ/cm2 and 150 mW/cm2, respectively, which is below the safety limits for human skin laser exposure in the near-infrared spectrum.

OA images were preferably acquired with a spherical matrix ultrasound array schematically depicted in FIG. 14a and described in detail above. The relative position and orientation of consecutive 3D images following an arbitrary scanning trajectory was expressed as a superposition of a translation and a rotation. To render a compounded OA volume, the rotation among subsequent acquisitions was estimated by cross-correlating their rotated Fourier spectra (FIG. 14b) whereas the translation fits were performed by a phase correlation method (FIG. 14c), as described in detail above.

FIG. 15 illustrates results of the spatial compounding procedure for the phantom scans. Clearly, the effective FOV in the vessel-mimicking phantom has been increased significantly by the spatial compounding procedure (FIG. 15c), further showing good agreement with the originally printed structure (FIG. 15a). Similarly, good agreement was found between the compounded OA image (FIG. 15f) and the bright-field microscopy image of the microsphere phantom (FIG. 15d). Note that, whereas the sphere location in the reconstructed OA images matches well the ground truth image, the average reconstructed size (˜300 μm) greatly exceeds the actual sphere diameter (100 μm). The discrepancy can be attributed to the convolution with the spatial resolution (point spread function) of the imaging system.

The results of the SVOT scan experiment are shown in FIG. 16. In this case, motion of the probe is fully calibrated by the translation and rotation stages so that accurate references are available for both orientation and position of each volumetric image frame (FIG. 16a). Due to the cylindrical scanning geometry, the motion estimations were further transformed into cylindrical coordinates with r, φ and z representing translation coordinates and θr, θφ and θz representing rotation angles with respect to the cylindrical axes. A subsequent analysis of the spatial compounding performance attained standard deviation (STD) of the translation estimation error of 1.29 voxels, 1.10 voxels and 1.06 voxels in the r, φ and z directions, respectively (isotropic voxel size is 100 μm). Note that translation in the r direction implies that only the distance of the probe from the object is altered, thus the imaged structures remain almost unaltered, i.e., the contribution of the d term in (7) is insignificant. Conversely, translation in the φ-z plane results in a more significant alteration of object's illumination and thus more significant alterations to the reconstructed image. This results in a less accurate translation estimation in the φ-z plane as compared to the r axis.

We further performed a quantitative comparison between rotation estimation accuracy with the 3D approach using (2) and the simplified 2D approach using (6), by considering that the actual positions of the array are known. The STD values of the error distributions (in degrees) are provided in the table in FIG. 16, suggesting that the 3D approach attains slightly more accurate results. However, this comes at the expense of significantly higher computational complexity. The estimation of rotation angles between two frames typically takes 0.08 s with the 2D approach versus 49.31 s with the 3D approach.

The 2D approach was therefore selected for compounding the entire mouse volume. Since the probe is solely rotated along the φ-axis during the SVOT scan, the orientation estimation and correction for the r and z axes was neglected. The translation estimation was performed using the two-step process, described in detail above. First, the position of frame iₙ was estimated based on the previous frame iₙ₋₁. Final adjustment was subsequently performed on the estimated position based on the stitched frame is. Moreover, the search range in the PoC was limited. FIG. 16b shows the known (blue dots) and estimated (red dots) positions of the spherical array probe in the r-φ plane for four exemplary slices along the z axis (exact location of the slices is labeled in panels f and g). While the estimation has proven to be accurate on a local scale, relatively large deviations between the known and estimated positions may occur due to accumulated errors. It should be noted that the image-based estimation of the trajectory of the probe may further depend on the animal motion during the scan (e.g. due to breathing), in which case the image-based spatial compounding may in fact partially compensate for the artifacts caused by motion of the imaged object. Nevertheless, a good agreement exists between the SVOT image attained using the proposed Fourier-based spatial compounding procedure (FIGS. 16d and 16g) and the corresponding (reference) image obtained by adding the frames using known locations and orientations of the array for all the scanning positions (FIGS. 16c and 16f). Yet, some blurring effects and loss of contrast can be recognized when comparing fine vascular structures in the images obtained using the estimated positions and orientations of the array (FIG. 16i) with the “ground truth” images rendered using known array positions (FIG. 16h). A direct comparison of the rendered images is provided in supplementary movie1. Quantitatively, the 3D rotation estimation method performs slightly better than the 2D estimation method, as summarized in the table shown in FIG. 16e.

FIG. 17 shows a schematic diagram illustrating volumetric optoacoustic angiography of a human arm performed in a zigzag-scan pattern. (a) Top and lateral maximum intensity projections of the compounded volume. Depth axis is color-coded. The white dashed line represents estimated the trajectory of the spherical array transducer during the scan. (b) Estimated single axis trajectories x(n), y(n) and z(n) of the transducer during the zigzag scan. The frame index n represents time via t=nT.

FIG. 17 illustrates results of the spatial compounding procedure for the zigzag scan. The top and lateral MIPs of the compounded volume are displayed in FIG. 17a as well as the projected estimated 3D trajectory, indicated by the white dashed line. The effective FOV has been increased significantly by the spatial compounding procedure from 15 mm×15 mm×15 mm (single volumetric frame) to 500 mm×500 mm×18 mm (compound volumetric frame). The inclination of the image and the projected trajectory in the x-y plane indicates that the orientation of the spherical matrix array does not exactly match the orientation of the translation path. Under this scenario, volumetric image compounding solely based on the known positions of the translation stage would result in severe misalignment artifacts. On the other hand, the Fourier-based compounding procedure is not affected by an insufficiently calibrated orientation, rendering accurately stitched volumes. Note that the spherical matrix array was moved along straight lines by the translation stage, yet some of the trajectories in FIG. 17a are bent despite the fact that the compound volume does not exhibit any signs of misalignment. This further suggests that the marginal motions of the human arm during the acquisition have been corrected implicitly by the spatial compounding procedure. Hence, the single axis trajectories x(n), y(n) and z(n) in FIG. 17b can be interpreted as motion superposition of both the spherical matrix array and the human arm, further emphasizing effectiveness of the developed spatial compounding procedure.

FIG. 18 shows a schematic diagram illustrating freehand optoacoustic human angiography of a human palm along an arbitrary trajectory. (a) Top and lateral maximum intensity projections of the compounded volume. Depth axis is color-coded. The white dashed line represents the projected 3D trajectory of the spherical array transducer operated in a freehand mode. (b) The estimated rotation angles during the scan (left) and the corresponding estimated transducer positions (right).

Results from the freehand scan of a human palm are showcased in FIG. 18. This experiment mimics a clinical imaging scenario where the transducer follows an arbitrary trajectory having all the six degrees of freedom. FIG. 18a shows MIPs of the compounded volume with the projected estimated 3D trajectory indicated by a dashed white line. In this example the effective FOV is increased from 12 mm×12 mm×12 mm (single volumetric frame) to approximately 50 mm×70 mm×15 mm (compound volume). The depth range was changed to cover the detected shift in the vertical (z) direction, which was approximately 3 mm. FIG. 18b shows the estimated transducer orientations for every frame in the three directions. The orientation remains predominantly constant with respect to the x and y axes whereas the z-axis orientation varies between −15° and +45°. The translation parameters are further estimated in FIG. 18c. The transducer was primarily moved along the x and y directions with the z-coordinate remaining nearly constant. This is expected considering the relatively flat skin surface in the imaged area. The compound volume appears to accurately represent an actual vascular network with no signs of misalignment perceived in the individual vessels. Note that, according to the estimated probe trajectory, some tissue areas were revisited during the scan. Yet, those areas appear seamlessly stitched despite the fact that they were compounded using non-consecutive frames.

The above approach relates to a universal methodology for spatial compounding of volumetric optoacoustic data acquired using either calibrated scanning systems or freehand-mode scans with up to six rotational and translational degrees of freedom. This is preferably accomplished by a purely image-based Fourier domain motion estimation method without using additional hardware for tracking the position and orientation of the detection array, which may turn challenging especially in the case of high-resolution freehand 3D scans. In particular, the combination of both rotation and translation estimation for volumetric image sequences is regarded as a novel and particularly advantageous aspect. Moreover, the approach outperforms feature-based techniques, which may often fail to provide sufficient reliability and accuracy due to an insufficient amount of feature matches in the registration step. Other registration methods, e.g. based on mutual information or sum of squared differences, may yet be considered.

The freehand real-time 3D imaging capacity greatly facilitates clinical utility of the OA imaging technology, with applications currently explored in many areas of clinical diagnostics of DMD, skin malignancies, breast tumors, vascular abnormalities, and inflammatory diseases, to name a few major examples. The fast imaging performance often comes at the cost of limited field-of-view, which can effectively be compensated by the developed trackerless spatial compounding algorithm.

The method's performance was validated by controlled experiments with phantoms, for which a reference image was available, and further supported by in vivo data acquired from mice and a human arm, where the positions of the transducer array were known exactly and served as a reference. In all cases the estimation accuracy remained below the diffraction-limited spatial resolution of the imaging system. Freehand scans of two phantoms and large-scale vascular trees in a human arm using arbitrary (unknown) trajectories have similarly rendered compounded image volumes having no visible signs of misalignments or discontinuities.

The algorithm performs optimally for inter-frame displacements in the order of 0.5-2 mm. Considering the ˜12×12 mm² effective field of view of the spherical array probe, this corresponds to >85% of voxels overlapping between two consecutive frames, thus ensuring they mainly contain similar structures. Accurate translation estimation may yet fail for larger displacements when significantly different images are rendered for consecutive positions or the images lack distinctive features. The maximum scanning speed then depends on the pulse repetition frequency of the laser. The latter is often kept in the 10-20 Hz range in order to conform to the safety limits pertaining average laser intensity on the human skin. A scanning speed below ˜2 cm/s would then be sufficient to guarantee the required overlap between consecutive images. Note that here we employed a simple superposition approach by simply adding up the image intensities values of the adjacent frames. In this case, non-uniform spatial sampling density due to e.g. varying motion velocity of the probe may lead to uneven signal intensities in the compounded images. Other compounding methods can alternatively be applied to facilitate accurate image formation, e.g. by weighting each voxel in the final image by the total number of scan frames effectively covering it. However, this would involve a much more significant modeling and computational effort to take into account the actual sensitivity field of the matrix array transducer, wavelength- and tissue-dependent volumetric light distribution in tissues etc. Another important consideration is the effectively covered imaging depth. In this regard, optical wavelengths experiencing weaker attenuation in tissues (e.g. 1064 nm) can be employed to expand the covered depth range, which may in turn facilitate image co-registration as more structures are visible in the images. However, it is additionally preferred to keep the depth range of interest within the field of view of the transducer during the entire freehand scan.

In the SVOT scan, the probe orientation between subsequent frames varied in the ±3° range with respect to all the three axes. In theory, the rotation estimation may remain accurate for arbitrarily large rotation angles, provided that the consecutive frames show sufficient overlap. In realistic optoacoustic imaging scenarios, however, an abrupt change in transducer orientation is accompanied by a very significant alteration of the effectively imaged field-of-view, resulting in lack of sufficient overlap between subsequent frames. These effects can be mitigated by using laser pulse repetition frequencies in the order of tens of Hz while ensuring that the probe is moved slowly and steadily during the freehand scan, avoiding abrupt leaps or changes in orientation. The experiments were executed using a relatively slow velocity not exceeding several centimeters per second. As a rule of thumb, the velocity scales linearly with the laser pulse repetition frequency and the effectively covered FOV. The accuracy of the motion estimation can be increased when employing higher resolution optoacoustic imaging systems. Recently, real-time 3D OA imaging with spatial resolution in the 35 μm range has been demonstrated using spherical matrix array transducer with 25 MHz usable detection bandwidth. In this case, the effective FOV is scaled down accordingly, thus reducing the permissible displacements between consecutive frames.

The developed methodology may enable accurate estimation of other types of motion not necessarily linked to freehand scanning. For instance, motion artefacts are often generated in a sequence of images due to breathing or heartbeat. In general, motion does not lead to blurring in single optoacoustic image frames since optoacoustic excitation is performed with very short (nanosecond duration) laser pulses while the responses are collected simultaneously by all the array elements. Nevertheless, many types of movements such as arterial pulsation is often accompanied by structural tissue deformations on a small spatial scale that cannot accurately be accounted for by assuming rigid motion. The latter approximation may still be valid in some cases where motion affects a region much larger than a single OA image volume where deformations remain minimal on a local scale (e.g. during breathing). In such cases, correcting for motion may help to better identify temporal changes in the signals, such as those associated with contrast agent perfusion or physiological activity. Even if no motion correction is performed, detection and rejection of the frames affected by motion may facilitate more efficient signal averaging, enhancing the spatial resolution and contrast-to-noise ratio of the images.

Motion correction is preferred for processing of MSOT data where displacements between the images acquired at different wavelengths may hinder accurate identification (unmixing) of spectrally-distinctive absorbers. Despite the fast wavelength tunability available with state-of-the-art pulsed lasers, even slight (sub-resolution) motion between images taken at different wavelengths may generate significant spectral unmixing artifacts in freehand scans.

The OA images may also be afflicted with artefacts related to acoustic scattering or reflections. While those were negligible in the present experiments solely involving soft tissues, areas including bones, lungs or other strongly mismatched regions may preferably be cropped before image registration.

In conclusion, a novel Fourier-based framework for spatial compounding of timelapse optoacoustic data acquired using large-area volumetric scans is disclosed herein. The method allows for rapidly combining sequential volumetric frames into large area scans without using additional tracking hardware. The new approach is universally applicable for compounding volumetric data acquired with calibrated scanning systems but also in a freehand mode with up to six rotational and translational degrees of freedom.

Preferably, the described framework or method is utilized for compounding 2D and/or volumetric optoacoustic images used with the devices, systems and methods for DMD diagnosis and/or analysis and/or monitoring described in detail above.

Robust performance was demonstrated for whole-body mouse imaging with spiral volumetric optoacoustic tomography as well as for freehand visualization of large-scale vascular networks in humans using 3D imaging probes. The newly introduced capability for angiographic observations at multiple spatial and temporal scales will greatly facilitate the use of optoacoustic imaging technology in pre-clinical research and clinical diagnostics, in particular DMD diagnostics. The volumetric compounding technique can equally benefit other biomedical imaging modalities looking at large-scale vascular network data, such as scanning fluorescence microscopy, optical coherence tomography or ultrasonography, thus optimizing their trade-offs between fast imaging performance and field-of-view.

1-16. (canceled)

17. A device for analyzing optoacoustic data comprising:
an interface for receiving optoacoustic data from a detection unit of an optoacoustic imaging system, wherein the optoacoustic data relate to acoustic waves that are generated in a tissue in response to irradiation of the tissue with time-varying electromagnetic radiation at two or more different irradiation wavelengths (λ), wherein the tissue comprises at least one of a muscle tissue, connective tissue, organ, tendon and/or pathogenic (fibrotic) tissue and
a processor to analyze the optoacoustic data to (i) determine a spatial distribution of at least one first value of an optoacoustic collagen signal (in a.u.), which relates to a concentration of collagen in the tissue, based on the optoacoustic data, (ii) derive at least one second value which corresponds to or is derived from at least one distribution parameter (optionally mean or max) characterizing the spatial distribution of the at least one first value of an optoacoustic collagen signal within a region of interest (ROI) of the spatial distribution of the at least one first value of an optoacoustic collagen signal, and (iii) provide the at least one second value and/or diagnostic information derived from the at least one second value for further use to a display unit for display.

18. The device according to claim 17, wherein the spatial distribution of the at least one first value of an optoacoustic collagen signal is two-dimensional or three-dimensional.

19. The device according to claim 17, wherein the at least one second value corresponds to or is derived from at least one of the following distribution parameters:
a mean value of the spatial distribution of the at least one first value of an optoacoustic collagen signal within the region of interest, and/or
a maximum value of the spatial distribution of the at least one first value of an optoacoustic collagen signal within the region of interest.

20. The device according to claim 19, wherein the at least one second value is derived from the mean value and the maximum value of the spatial distribution of the at least one first value of an optoacoustic collagen signal within the region of interest.

21. The device according to claim 19, wherein the at least one second value corresponds to a ratio between the mean value and the maximum value of the spatial distribution of the at least one first value of an optoacoustic collagen signal within the region of interest.

22. The device according to claim 17, wherein the processor is further configured to derive the diagnostic information from the at least one second value by comparing the at least one second value with at least one predefined reference value.

23. The device according to claim 17, wherein the derived diagnostic information relates to the presence or absence and/or likelihood of the presence or absence of a muscle disorder in the tissue, wherein the muscle disorder is optionally Duchenne muscular dystrophy (DMD).

24. The device according to claim 17, wherein the processor is further configured to
reconstruct an ultrasound image of the tissue based on ultrasound data (detector signals) relating to ultrasound waves reflected by the tissue in response to ultrasound waves impinging on the tissue, and
provide the ultrasound image of the tissue for displaying the ultrasound image of the tissue on a display unit.

25. The device according to claim 17, wherein the display unit is configured to display information, and wherein the processor is further configured to control the display unit to display
the spatial distribution of the at least one first value of an optoacoustic collagen signal, which relates to a concentration of collagen in the tissue, and/or
the ultrasound image of the tissue, and/or
the at least one second value, and/or
the diagnostic information derived from the at least one second value.

26. The device according to claim 25, wherein the processor is further configured to merge the spatial distribution of the at least one first value of an optoacoustic collagen signal, which relates to a concentration of collagen in the tissue, and the ultra-sound image of the tissue to obtain a merged optoacoustic-ultrasound image of the tissue, and to control the display unit to display the merged optoacoustic-ultrasound image of the tissue.

27. The device according to claim 25, wherein the display unit and/or the processor is further configured to enable a user to select the region of interest (ROI), within which the at least one second value is derived from the spatial distribution of the at least one first value, in the displayed spatial distribution of the at least one first value and/or in the displayed merged optoacoustic-ultrasound image of the tissue.

28. An optoacoustic system for generating and analyzing optoacoustic data, the system comprising
an irradiation unit configured to irradiate a tissue comprising muscle tissue with electromagnetic radiation at two or more different irradiation wavelengths (λ), said electromagnetic radiation having a time-varying, optionally pulsed, intensity,
a detection unit configured to detect acoustic waves generated in the tissue in response to irradiating the tissue with the electromagnetic radiation at the different irradiation wavelengths (λ) and to generate according optoacoustic data, and
the device for analyzing optoacoustic data according to claim 17.

29. The system according to claim 28, wherein the irradiation unit is configured to irradiate the tissue with electromagnetic radiation at two or more different irradiation wavelengths (λ) being in a wavelength range between 650 nm and 1200 nm, optionally between 680 nm and 1100 nm.

30. A method for analyzing optoacoustic data, the method comprising the following steps:
receiving optoacoustic data (detector signals) relating to acoustic waves generated in the tissue in response to irradiating the tissue with time-varying electromagnetic radiation at two or more different irradiation wavelengths (λ)
determining a spatial distribution of at least one first value (optoacoustic collagen signal in a.u.), which relates to a concentration of collagen in tissue comprising at least one of a muscle tissue, connective tissue, organ, tendon and/or pathogenic (fibrotic) tissue, and is based on the optoacoustic data,
deriving at least one second value from the spatial distribution of the at least one first value, the at least one second value corresponding to or being derived from at least one distribution parameter (optionally mean or max) characterizing the spatial distribution of the at least one first value within a region of interest of the spatial distribution of the at least one first value, and
providing the at least one second value and/or diagnostic information derived from the at least one second value to a display unit for display.

31. A computer program product which causes a computer to execute the method according to claim 30.

32. A method of diagnosing a muscle disorder, optionally Duchenne muscular dystrophy (DMD), in a patient, comprising:
irradiating a tissue in the patient with time-varying electromagnetic radiation at two or more different irradiation wavelengths (λ), wherein the tissue comprises at least one of a muscle tissue, connective tissue, organ, tendon and/or pathogenic (fibrotic) tissue,
determining a spatial distribution of at least one optoacoustic collagen signal (optionally in arbitrary units a.u.), which relates to a concentration of collagen in the tissue based on optoacoustic data (detector signals) relating to acoustic waves generated in the tissue in response to irradiating the tissue,
deriving at least one second value, which corresponds to or is derived from at least one distribution parameter characterizing the spatial distribution of the at least one optoacoustic collagen signal within a region of interest of the spatial distribution of the at least one optoacoustic collagen signal, and
outputting, optionally displaying on a display unit, the at least one second value and/or diagnostic information which has been derived from the at least one second value by comparing the at least one second value with at least one predefined reference value and which relates to the presence or absence and/or likelihood of the presence or absence of a muscle disorder, optionally Duchenne muscular dystrophy (DMD), in the tissue of the patient.

33. A method of treating a muscle disorder in a patient, optionally Duchenne muscular dystrophy (DMD), comprising:
irradiating a tissue in the patient with time-varying electromagnetic radiation at two or more different irradiation wavelengths (λ), wherein the tissue comprises at least one of a muscle tissue, connective tissue, organ, tendon and/or pathogenic (fibrotic) tissue,
determining a spatial distribution of at least one optoacoustic collagen signal (optionally in arbitrary units a.u.), which relates to a concentration of collagen in the tissue based on optoacoustic data (detector signals) relating to acoustic waves generated in the tissue in response to irradiating the tissue,
deriving at least one second value, which corresponds to or is derived from at least one distribution parameter characterizing the spatial distribution of the at least one optoacoustic collagen signal within a region of interest of the spatial distribution of the at least one optoacoustic collagen signal, and
outputting, optionally displaying on a display unit, the at least one second value and/or diagnostic information which has been derived from the at least one second value by comparing the at least one second value with at least one predefined reference value and which relates to the presence or absence and/or likelihood of presence or absence of a muscle disorder, optionally Duchenne muscular dystrophy (DMD), in the tissue of the subject, and
treating the subject, optionally by administering a medication to the subject, in accordance with the diagnostic information.

34. A method of treatment as described in claim 33, wherein the course of treatment depends on but is not limited to:
selecting the medication and/or an active agent of the medication depending on the at least one second value and/or diagnostic information,
selecting the dose of an active agent contained in the medication depending on the at least one second value and/or diagnostic information,
selecting time and/or duration of administering the medication depending on the at least one second value and/or diagnostic information.

35. A method of treatment as described in claim 34, wherein treating the subject with a medication comprises:
administering at least one corticosteroid, optionally Deflazacort and/or Ataluren (Translarna™, PTC Therapeutics Inc.) and/or Spinraza® (Nusinersen) and/or Zolgensma® (Onasemnogene abeparvovec) or other approved drugs, and/or
administering one or more of Eteplirsen (Exondys 51®), corticosteroids, such as prednisone, and/or heart medications, such as angiotensin-converting enzyme (ACE) inhibitors or beta blockers.
